(19) United States
(12) Patent Application Publication
(10) Pub. No.: US 2012/0164110 A1
(43) Pub. Date: Jun. 28, 2012

(54) DIFFERENTIALLY METHYLATED REGIONS OF REPROGRAMMED INDUCED PLURIPOTENT STEM CELLS, METHOD AND COMPOSITIONS THEREOF

(76) Inventors: Andrew P. Feinberg, Lutherville, MD (US); George Q. Daley, Weston, MA (US)

(21) Appl. No.: 13/184,426

(22) Filed: Jul. 15, 2011

Related U.S. Application Data
(63) Continuation-in-part of application No. PCT/US2010/033281, filed on Apr. 30, 2010.
(60) Provisional application No. 61/251,467, filed on Oct. 14, 2009.
(60) Provisional application No. 61/306,707, filed on Feb. 22, 2010.
(60) Provisional application No. 61/365,279, filed on Jul. 16, 2010.

(51) Int. Cl. A61K 35/12; C12N 5/071; C40B 40/06; C12Q 1/68; C40B 20/00

(52) U.S. Cl. 424/93.7; 435/6.11; 506/2; 506/16; 435/377

(57) ABSTRACT

Provided herein are differentially methylated regions (DMRs) of reprogrammed iPS cells (R-DMRs) and methods of use thereof. The invention provides methods for detecting and analyzing alterations in the methylation status of DMRs in iPS cells, somatic cells and embryonic stem (ES) cells as well as methods for reprogramming somatic cells to generate an iPS cell.

### RELATED APPLICATION DATA

This application is a Continuation-in-part application of International Application No. PCT/US2010/033281, filed Apr. 30, 2010, which claims the benefit of priority under 35 U.S.C. §119(e) of U.S. Provisional Patent Application Ser. No. 61/251,467, filed Oct. 14, 2009; and the benefit of priority under 35 U.S.C. §119(e) of U.S. Provisional Patent Application Ser. No. 61/306,707, filed Feb. 22, 2010, the entire content of which are incorporated herein by reference. Additionally, this application claims the benefit of priority under 35 U.S.C. §119(e) of U.S. Provisional Patent Application Ser. No. 61/365,279, filed Jul. 16, 2010, the entire content of which is incorporated herein by reference in entirety.

### STATEMENT OF GOVERNMENT SUPPORT

This invention was made in part with government support under Grant Nos. P50HG003233-06, R37CA054358, RO1-DK70055, RO1-DK59279, RC2-HL102815, K99HL093212-01, R01AI047457, R01AI047458, CA86065, and HL099999 awarded by the National Institutes of Health. The United States government has certain rights in this invention.

### BACKGROUND OF THE INVENTION

1. Field of the Invention

The present invention relates generally to differentially methylated regions (DMRs) in the genome outside CpG islands in induced pluripotent stem (iPS) cells, and more specifically to methods for detecting and analyzing alterations in the methylation status of DMRs in iPS cells, somatic cells and embryonic stem (ES) cells as well as methods for reprogramming somatic cells to generate an iPS cell.

2. Background Information

Epigenetics is the study of non-sequence information of chromosome DNA during cell division and differentiation. The molecular basis of epigenetics is complex and involves modifications of the activation or inactivation of certain genes. Additionally, the chromatin proteins associated with DNA may be activated or silenced. Epigenetic changes are preserved when cells divide. Most epigenetic changes only occur within the course of one individual organism's lifetime, but some epigenetic changes are inherited from one generation to the next.

One example of an epigenetic mechanism is DNA methylation (DNAm), a covalent modification of the nucleotide cytosine. In particular, it involves the addition of methyl groups to cytosine nucleotides in the DNA, to convert cytosine to 5-methylcytosine. DNA methylation plays an important role in determining whether some genes are expressed or not. Abnormal DNA methylation is one of the mechanisms known to underlie the changes observed with aging and development of many cancers.

It has been shown that alterations in DNA methylation (DNAm) occur in cancer, including hypomethylation of oncogenes and hypermethylation of tumor suppressor genes. However, most studies of cancer methylation assumed that functionally important DNAm will occur in promoters, and that most DNAm changes in cancer occur in CpG islands. However, it was determined that most methylation alterations in certain cancers occur not in promoters, and also not in CpG islands, but in sequences up to 2 kb distant, which are termed ‘CpG island shores’. Differential methylation patterns that distinguish among normal tissue types (T-DMRs) and patterns that can segregate colorectal cancer tissue from matched normal tissues (C-DMRs) have been described. Unexpectedly, these two DMRs occur 13-fold more frequently at CpG island ‘shores’, regions of comparatively low CpG density that are located near traditional CpG islands, than at the CpG islands themselves. Cancers showed approximately equal numbers of hypomethylated and hypermethylated regions, and 45% of C-DMRs overlapped T-DMRs, suggesting that epigenetic changes in cancer involve reprogramming of the normal pattern of tissue-specific differentiation.

iPS cells are derived by epigenetic reprogramming. For example, iPS cells can be derived from somatic cells by introduction of a small number of genes: for example, POU5F1, MYC, KLF4 and SOX2. As direct derivatives of an individual's own tissue, iPS cells offer considerable therapeutic promise, avoiding both immunologic and ethical barriers to their use. iPS cells differ from their somatic parental cells epigenetically, and thus a comprehensive comparison of the epigenome in iPS and somatic cells would provide insight into the mechanism of tissue reprogramming. Although two previous targeted studies examined a subset of the genome, 7,000 (Ball et al. (Nat. Biotechnol. (27)485 (2009)) and 66,000 (Deng et al. (Nat. Biotechnol. (27)353-360 (2009))) CpG sites in a small cohort of three iPS-fibroblast pairs, a global assessment of genome-wide methylation has not yet been performed.

Direct reprogramming of somatic cells with the transcription factors Oct4, Sox2, K1f4, and c-Myc yields induced pluripotent stem cells (iPSC) with striking similarity to embryonic stem cells from fertilized embryos (fESC). Like fESC, iPSC form teratomas, differentiated tumors with tissues from all three embryonic germ layers, and when injected into murine blastocysts contribute to all tissues, including the germ line. iPSC from mouse embryo fibroblasts generate “all-iPSC mice” following injection into tetraploid blastocysts, thereby satisfying the most stringent criterion of pluripotency. Embryonic tissues are the most efficiently reprogrammed, producing iPSC that are nearly identical to fESC. In contrast, reprogramming from accessible adult tissues, most applicable for modeling diseases and generating therapeutic cells, is inefficient and limited by barriers related to the differentiation state and age of the donor's cells. Aged cells have higher levels of Ink4/Arf, which limits the efficiency and fidelity of reprogramming. Moreover, terminally differentiated blood cells reprogram less efficiently than blood progenitors. As with cloning by nuclear transfer in frogs and mice, the efficiency and yield of reprogrammed genomes declines with increasing age and differentiation status of the donor cell, and varies with the methylation state of the donor nucleus.

Different tissues show variable susceptibility to reprogramming. Keratinocytes reprogram more readily than fibroblasts, and iPSC from stomach or liver cells harbor fewer integrated proviruses than fibroblasts, suggesting they require lower levels of the reprogramming factors to achieve pluripotency. When differentiated into neurospheres, iPSC from adult tail-tip fibroblasts retain more teratoma-forming cells than iPSC from embryonic fibroblasts, again indicating heterogeneity based on the tissue of origin. Moreover, cells can exist in intermediate states of reprogramming that interconvert with continuous passage or treatment with chromatin-modifying agents. Although generic iPSC are highly similar to fESC, in practice iPSC generated from various tissues may harbor significant differences, both functional and molecular which has yet to be determined.

### SUMMARY OF THE INVENTION

The present invention is based on the discovery that alterations in DNA methylation in iPS cells, as compared to both ES cells and parental fibroblasts, occur not only in promoters or CpG islands, but in sequences up to 2 kb distant from such CpG islands (such sequences are termed “CpG island shores”). In accordance with this discovery, there are provided herein DMR of reprogrammed iPS cells (R-DMRs) and methods of use thereof.

In one aspect of the invention, there is provided a method of generating an iPS cell. The method includes contacting a somatic cell with an agent that alters the methylation status of one or more nucleic acid sequences of the somatic cell, the one or more nucleic acid sequences being outside of a promoter region of a gene and outside of a CpG island, and wherein the nucleic acid sequences are up to about 2 kb in distance from a CpG island, and wherein the nucleic acid sequences are differentially methylated in reprogrammed somatic cells as compared with parent somatic cells, thereby generating an iPS cell. In certain embodiments, the one or more nucleic acid sequences are any combination of DMR sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof. In certain embodiments, the method further comprises detecting the methylation status profile of the one or more nucleic acid sequences of the induced iPS. In yet another aspect, the method further comprises comparing the methylation status profile to a methylation status profile of the one or more nucleic acid sequences of a parental somatic cell from which the iPS is induced.

In particular embodiments, the agent is a nuclear reprogramming factor. In various embodiments, the nuclear reprogramming factor is a nucleic acid encoding a SOX family gene, a KLF family gene, a MYC family gene, POU5F1, SALL4, OCT4, NANOG, LIN28, or the expression product thereof. For example, in exemplary embodiments, the nuclear reprogramming factor is one or more of POU5F1, OCT4, SOX2, KLF4, or C-MYC.

In another aspect, there is provided an iPS cell produced using the methods of the invention.

In another aspect, there is provided a population of iPS cells produced using the methods the invention.

In yet another aspect of the invention, there is provided a plurality of nucleic acid sequences, wherein the nucleic acid sequences are outside of a promoter region of a gene and outside of a CpG island, and wherein the nucleic acid sequence is up to about 2 kb in distance from a CpG island, and wherein the nucleic acid sequences are differentially methylated in the reprogramming of a somatic cell to generate an iPS cell. In some embodiments, the nucleic acid sequence are any DMR sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, and the IGF1R gene. In one embodiment, the plurality of nucleic acid sequences is a microarray.

In another aspect of the invention, there is provided a plurality of nucleic acid sequences, wherein the nucleic acid sequences are outside of a promoter region of a gene and outside of a CpG island, and wherein the nucleic acid sequences are up to about 2 kb in distance from a CpG island, and wherein the methylation status of the nucleic acid sequences is altered in an iPS cell as compared to an ES cell. In some embodiments, the nucleic acid sequence are any DMR sequences as set forth in Table 7, FIGS. 4C-4G, the PTPRT gene, the POU3F4 gene, the AZBP1 gene, the ZNF184 gene, and the IGF1R gene. In one embodiment, the plurality of nucleic acid sequences is a microarray.

In yet another aspect of the invention, there is provided a method of identifying an iPS cell. The method includes comparing the methylation status of one or more nucleic acid sequences of a putative iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of an iPS cell, wherein a similarity in methylation status is indicative of the putative cell being an iPS cell. In certain embodiments, the one or more nucleic acid sequences are DMR sequences as set forth in Tables 2, 6, 7, 9, FIGS. 1B-1C, FIGS. 4C-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof.

In yet another aspect of the invention, there is provided a method of identifying an iPS cell. The method includes comparing the methylation status of one or more nucleic acid sequences of a putative iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of a corresponding somatic cell from which the iPS cell is induced or ES cell, wherein an alteration in methylation status is indicative of the putative cell being an iPS cell. In certain embodiments the method further includes comparing the methylation status of the one or more nucleic acid sequences of the putative iPS cell to a known methylation status of the one or more nucleic acid sequences of a corresponding somatic cell from which the iPS cell is induced. In such embodiments, the one or more nucleic acid sequences are DMR sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof. In certain embodiments the method further includes comparing the methylation status of the one or more nucleic acid sequences of the putative iPS cell to the methylation status of the one or more nucleic acid sequences of a known ES cell. In such embodiments, the one or more nucleic acid sequences are DMR sequences as set forth in Table 6, FIGS. 4C-4G, the PTPRT gene, the POU3F4 gene, the AZBP1 gene, the ZNF184 gene, the IGF1R gene, and any combination thereof.

In various aspect of the invention, the one or more nucleic acid sequences are within a gene. Alternatively, the one or more nucleic acid sequences are upstream or downstream of a gene. In various embodiments, determination of methylation status is performed by one or more techniques selected from the group consisting of a nucleic acid amplification, polymerase chain reaction (PCR), methylation specific PCR, bisulfate pyrosequencing, single-strand conformation polymorphism (SSCP) analysis, restriction analysis, microarray technology, and proteomics.

In yet another aspect of the invention, there is provided a method for providing a methylation map of a region of genomic DNA isolated from an iPS cell. The method includes performing comprehensive high-through array-based relative methylation (CHARM) analysis on a sample of labeled, digested genomic DNA isolated from the iPS cell, thereby providing a methylation map for the iPS cell. In certain embodiments, the method further includes performing one or more techniques, such as a nucleic acid amplification, polymerase chain reaction (PCR), methylation specific PCR, bisulfite pyrosequencing, single-strand conformation polymorphism (SSCP) analysis, and restriction analysis.

In yet another aspect of the invention, there is provided a method of characterizing the methylation status of the nucleic acid of an iPS cell. The method includes a) hybridizing labeled and digested nucleic acid of an iPS cell to a DNA microarray comprising at least 2000 nucleic acid sequences, with the proviso that the nucleic acid sequences are outside of a promoter region of a gene and outside of a CpG island, and wherein the nucleic acid sequences are up to about 2 kb in distance from a CpG island; and b) determining a pattern of methylation from the hybridizing of (a), thereby characterizing the methylation status for the iPS cell. In particular embodiments, the one or more nucleic acid sequences are DMR sequences as set forth in Tables 2, 6, 7, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, the IGF1R gene and any combination thereof. In certain embodiments, the method further includes comparing the methylation status profile to a methylation profile from hybridization of the microarray with labeled and digested nucleic acid from a parental somatic cell from which the iPS is induced or from an ES cell.

In yet another embodiment, there is provided a method of treating a subject. The method includes a) obtaining a somatic cell from a subject; b) reprogramming the somatic cell into an iPS cell using the methods of the invention; c) culturing the pluripotent stem (iPS) cell to differentiate the cell into a desired cell type suitable for treating a condition; and d) introducing into the subject the differentiated cell, thereby treating the condition.

In yet another aspect of the invention, there is provided a method of enhancing the differentiation potential of an induced pluripotent stem (iPS) cell. The method includes contacting an iPS cell with a demethylating agent, thereby reducing the epigenetic memory of the iPS cell as compared to the epigenetic memory of the iPS cell prior to contact with the demethylating agent, thereby enhancing the differentiation potential of an iPS cell as compared with a cell not contacted with a demethylating agent. In various embodiments, the iPS cell is generated by contact with a nuclear reprogramming factor, such as, but not limited to one or more of POU5F1, OCT4, SOX2, KLF4, or C-MYC. In various embodiments, the demethylating agent may be any known demethylating agent. For example, the demethylating agent may be a DNA (cytosine-5)-methyltransferase 1 (DNMT1) inhibitor or a cytidine analog, such as 5-azacytidine, 5-aza-2-deoxycytidine. Another example includes zebularine. In various embodiments, the method may further include contacting the cell with a histone deacetylase (HDAC) inhibitor, such as trichostatin A.

In yet another aspect of the invention, there is provided a method of enhancing the differentiation potential of an induced pluripotent stem (iPS) cell. The method a) differentiating a first iPS cell generated from a first cell lineage into a cell of a second cell lineage, wherein the first and second cell lineages are different; and b) generating a second iPS cell from the differentiated cell of a), thereby altering the epigenetic memory of the first iPS cell as compared to the epigenetic memory of the second iPS cell, thereby enhancing the differentiation potential of the second iPS cell as compared with the first iPS cell. In various embodiments, the method may further include performing methylome analysis on one or more of the cells of (a) or (b). In various embodiments, the first or second iPS cell is generated by contact with a nuclear reprogramming factor, such as, but not limited to one or more of POU5F1, OCT4, SOX2, KLF4, or C-MYC. In various embodiments, the method may further include contacting the first or second iPS cell with a demethylating agent. In various embodiments, the demethylating agent may be any known demethylating agent. For example, the demethylating agent may be a DNA (cytosine-5)-methyltransferase 1 (DNMT1) inhibitor or a cytidine analog, such as 5-azacytidine, 5-aza-2-deoxycytidine. Another example includes zebularine. In various embodiments, the method may further include contacting the cell with a histone deacetylase (HDAC) inhibitor, such as trichostatin A.

In yet another aspect of the invention, there is provided a method of differentiating an induced pluripotent stem (iPS) cell. The method may include a) contacting an iPS cell with a demethylating agent; and b) contacting the cell of a) with a differentiation factor, thereby differentiating the iPS cell. In various embodiments, the method may further include performing methylome analysis on one or more of the cells of (a) or (b). In various embodiments, the iPS cell is generated by contact with a nuclear reprogramming factor, such as, but not limited to one or more of POU5F1, OCT4, SOX2, KLF4, or C-MYC. In various embodiments, the method may further include contacting iPS cell with a demethylating agent. In various embodiments, the demethylating agent may be any known demethylating agent. For example, the demethylating agent may be a DNA (cytosine-5)-methyltransferase 1 (DNMT1) inhibitor or a cytidine analog, such as 5-azacytidine, 5-aza-2-deoxycytidine. Another example includes zebularine. In various embodiments, the method may further include contacting the cell with a histone deacetylase (HDAC) inhibitor, such as trichostatin A.

In yet another aspect of the invention, there is provided a method of differentiating an induced pluripotent stem (iPS) cell. The method includes a) differentiating a first iPS cell generated from a first cell lineage into a cell of a second cell lineage, wherein the first and second cell lineages are different; b) generating a second iPS cell from the differentiated cell of a); and c) contacting the second iPS cell with a differentiation factor, thereby differentiating the iPS cell. In various embodiments, the method may further include performing methylome analysis on one or more of the cells of (a) to (c). In various embodiments, the first or second iPS cell is generated by contact with a nuclear reprogramming factor, such as, but not limited to one or more of POU5F1, OCT4, SOX2, KLF4, or C-MYC. In various embodiments, the method may further include contacting the first or second iPS cell with a demethylating agent. In various embodiments, the demethylating agent may be any known demethylating agent. For example, the demethylating agent may be a DNA (cytosine-5)-methyltransferase 1 (DNMT1) inhibitor or a cytidine analog, such as 5-azacytidine, 5-aza-2-deoxycytidine. Another example includes zebularine. In various embodiments, the method may further include contacting the cell with a histone deacetylase (HDAC) inhibitor, such as trichostatin A.

In yet another aspect of the invention, there is provided a method of identifying the differentiation potential of an induced pluripotent stem (iPS) cell. The method includes comparing the methylation status of one or more nucleic acid sequences of an iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of a reference iPS cell or a non-induced pluripotent stem cell, wherein a similarity or a difference in methylation status between the iPS cell and the reference iPS cell or the non-induced pluripotent stem cell is indicative of the differentiation potential of the iPS cell. In some embodiments, the one or more nucleic acid sequences are within a gene. In some embodiments, the one or more nucleic acid sequences are upstream or downstream of a gene. In various embodiments, the one or more nucleic acid sequences are selected from differentially methylated region (DMR) sequences as set forth in Tables 14, 15, 12, FIGS. 10, 14, 17, 18, the POU5F1 gene, the NANOG gene, the OCT4 gene, the SOX2 gene, the KLF4 gene, the C-MYC gene and any combination thereof.

In yet another aspect of the invention, there is provided a method of modifying the lineage restriction of a pluripotent stem (PS) cell. The method includes contacting a PS cell with an agent which alters regulation of the expression or expression product of a gene known to be associated with the differentiation potential of the PS cell, thereby modifying the lineage restriction of the PS cell. In various embodiments, the agent alters regulation of the expression or expression product of a gene set forth in Tables 14, 15, 12, FIGS. 10, 14, 17, 18, the POU5F1 gene, the NANOG gene, the OCT4 gene, the SOX2 gene, the KLF4 gene, the C-MYC gene and any combination thereof. In various embodiments, the agent is a demethylating agent. The demethylating agent may be a DNA (cytosine-5)-methyltransferase 1 (DNMT1) inhibitor or a cytidine analog, such as 5-azacytidine, 5-aza-2-deoxycytidine. Another example includes zebularine. In various embodiments, the method may further include contacting the cell with a histone deacetylase (HDAC) inhibitor, such as trichostatin A. In various embodiments, the agent is a vector comprising a nucleic acid sequence encoding a gene or portion thereof; a polynucleotide, such as an antisense oligonucleotides including microRNA, dsRNA, siRNA, stRNA, and shRNA; a polypeptide, or a small molecule.

In yet another aspect of the invention, there is provided a method of generating a cell bank. The method includes a) identifying the differentiation potential of a plurality of pluripotent stem (PS) cells; and b) sorting the cells of (a) by differentiation potential.

In yet another aspect of the invention, there is provided a cell bank produced by a method of the invention.

In yet another aspect of the invention, there is provided a method of treating a subject, the method including a) diagnosing a subject to determine a disease or a disorder; b) generating a plurality of pluripotent stem (PS) cells; c) analyzing the plurality of PS cells to determine a differentiation potential for an individual stem cell of the plurality; d) isolating an individual stem cell of (c) based on the disease or disorder of (a); and e) introducing into the subject the stem cell of (d), thereby treating the disease or the disorder.

### BRIEF DESCRIPTION OF THE DRAWINGS

FIG. 1A shows plots depicting the distribution of distance of R-DMRs from CpG islands. FIGS. 1B and 1C, upper panels, show plots of M value versus genomic location for fibroblast and iPS cells and plots of CpG density versus genomic location, where the curve represents averaged smoothed M values; the location of CpG dinucleotides (black tick marks), CpG density, location of CpG islands (filled boxes along the x-axis (zero)), along with the gene annotation. FIGS. 1B and 1C, lower panels show validation by bisulfate pyrosequencing of methylation percentage mapping to the unfilled box along the x-axis of the plots of CpG density versus genomic location in the upper panels of FIGS. 1B and 1C for various iPS cells, fibroblasts, ES cells (BGO1, BGO3 and H9) as well as the highly methylated HCT116 colon cancer cell line and a generally hypomethylated double DNA methyltransferase 1/3B double knockout line (DKO) derived from it.

FIG. 2 shows plots depicting the distribution of distance of R-DMRs from CpG islands.

FIG. 3A shows a clustering of M values of all tissues from the 4,401 regions (FDR<0.05) corresponding to R-DMRs (iPS cells compared to parental fibroblasts) comparing normal brain, spleen and liver tissues (denoted as Br, Sp and Lv, respectively). FIG. 3B shows a clustering of M values of all tissues from the 4,401 regions (FDR<0.05) corresponding to R-DMRs (iPS cells compared to parental fibroblasts) comparing colorectal cancer and matched normal colonic mucosa (denoted as T and N, respectively).

FIG. 4 shows plots depicting differential DNA methylation (upper panels) and confirmation by bisulfite pyrosequencing (lower panels) for DMRs found by comparison between iPS cells and fibroblasts (A and B) as well as various genes (C-G). FIGS. 4A-G, upper panels, show plots of M value versus genomic location, where the curve represents averaged smoothed M values. Also shown in the upper panels are the location of CpG dinucleotide (black tick marks on x-axis), CpG density (smoothed black line) calculated across the region using a standard density estimator, location of CpG islands (filled boxes along the x-axis (zero)), as well as gene annotation indicating the transcript (thin outer gray line), coding region (thin inner gray line), exons (filled gray box) and gene transcription directionality on the y-axis (sense marked as +, antisense as −). FIGS. 4A-G, lower panels depict plots showing the degree of DNA methylation as measured by bisulfite pyrosequencing. The unfilled box indicated on the x-axis of the CpG density plot in the upper panel indicates the CpG sites that were measured. Reactions were performed in triplicate; bars represent the mean methylation±SD of iPS cells, fibroblasts, and ES cells (BGO1, BGO3 and H9) as well as DKO (DNMT1 and DNMT3B Double KO cell line) and HCT116 (parental colon cancer cell line) for each individual CpG site measured.

FIG. 5 shows plots of differential gene expression versus differential methylation for R-DMRs at CpG island shores.

FIG. 6 is a pictorial representation of an experimental schema. Experimental schema. fESC, ntESC, F-iPSC, and B-iPSC were derived from B6/CBA F1 mice by reprogramming and/or cell culture, characterized for pluripotency by criteria applied to human cells, followed by differentiation analysis for osteogenic or hematopoietic lineages.

FIG. 7 is a series of graphical representations depicting differentiation of cell lines. FIG. 7A is a plot of hematopoietic colony number per 100,000 EB cells differentiated from indicated cell lines. FIG. 7B is a plot of quantification of elemental calcium by inductively coupled plasma—atomic emission spectroscopy in 5×10⁵ cells after osteogenic differentiation of indicated cell lines. FIG. 7C is a plot of Q-PCR of osteogenic genes, Bglap, Sp7, and Runx2 in indicated cell lines after osteogenic differentiation. Gene expression was normalized to Actin. n=number of independent clones tested. Error bars=s.d.

FIG. 8 is a series of graphical representations of analysis of methylation in stem cell lines. FIG. 8A is a cluster dendrogram using probes from DMRs that distinguish B-iPSC and F-iPSC. Cell clones are described in Table 17. FIG. 8B are graphical plots of enrichment of DMRs for hematopoiesis and fibroblast-related transcription factors in B-iPSC and F-iPSC, relative to chance (100,000 random permutations). The left panel of FIG. 8B shows that 20 of 74 hematopoiesis-related transcription factors overlap DMRs hypermethylated in F-IPSC (p=0.0034). The right panel of FIG. 8B shows that 115 of 764 fibroblast-specific genes overlap DMRs hypermethylated in B-iPSC (p=10⁻⁵).

FIG. 9 is a series of pictorial and graphical representations showing stringently-defined pluripotent stem cells and their characterization. FIG. 9A is an experimental schema. Four horizontal lines indicate integrated proviruses carrying dox-inducible reprogramming factors in some experiments. Characteristics of individual clones in all subsequent panels can be found in Table 17. FIG. 9B is a plot of hematopoietic colony number per 100,000 EB cells differentiated from indicated cell lines. n=number of independent clones tested. Error bars=s.d., added for clones repeated three or more times. FIG. 9C is a cluster dendrogram using probes from DMRs that distinguish Bl-iPSC and NP-iPSC.

FIG. 10 is a series of graphical representations of examples of differential DNA methylation (upper panels) and confirmation by bisulfite pyrosequencing (lower panels). The upper panel is a plot of p (percent methylation) value versus genomic location, where the curve represents averaged smoothed p values. The location of CpG dinucleotide (black tick marks on x axis), CpG density (smoothed black line) calculated across the region using a standard density estimator, location of CpG islands (shown on X axis of CpG density panel), as well as gene annotation indicating the transcript (thin outer gray line), coding region (thin inner gray line), exons (filled gray box) and gene transcription directionality on the y axis (sense marked as +, antisense as −) are also shown in the upper panels. The lower panel represents the degree of DNA methylation as measured by bisulfite pyrosequencing. The box indicated on the x axis of the CpG density plot in the upper panels indicates the CpG sites that were measured. FIG. 10A is for Slc32a1. FIG. 10B is for Cd37. FIG. 10C is for Rest. FIG. 10D is for Kcnrg.

FIG. 11 is a series of graphical representations of gene enrichment analysis of DMRs. FIG. 11A shows enrichment of liver-related genes in differentially methylated regions (DMRs) between B-iPSC and F-iPSC as a negative control. FIG. 11B shows enrichment of neural-related genes in DMRs that distinguish Bl-iPSC from NP-iPSC. FIG. 11C is a plot showing gene enrichment analysis in which higher than expected overlap of hematopoietic-specific genes with DMRs hypomethylated in TSA-AZA-treated NP-iPSC versus NP-iPSC. Thick vertical line indicates overlap of 63 such genes out of 526 interrogated. Grey histogram represents a random probability distribution of overlap (P=0.0012; 100,000 permutations).

FIG. 12 is a series of pictorial and graphical representations showing analysis of methylation in stem cell lines. FIG. 12A is a cluster dendrogram analysis of B-iPSC and Bl-iPSC with hematopoetic lineage progenitors (MPP: multipotent progenitors, CLP: common lymphoid progenitors, and CMP: common myeloid progenitors). Unsupervised, average linkage cluster analysis was performed using Euclidian distance based on the probes in the regions that intersect between the CMP vs CLP DMRs and the B-iPSC vs Bl-iPSC DMRs. FIG. 12B is a cluster dendrogram analysis using the probes in the regions that have differential methylation between fibroblast and bone marrow from B6CBA and B6129 mice.

FIG. 13 is a series of pictorial representations of heat maps. Overlap of DMRs with loci of genes showing fESC-specific gene expression (determined from compiled microarray data. Heat maps reflect expression values of fESC-specific genes in undifferentiated state (fESC D0; top 5% highly expressed genes; 554 genes) and after differentiation for 2 and 9 days (differentiated fESC day 2; dfESC D2 and day 9; dfESC D9). FIG. 13A is a heat map with grey bars in the right three lanes indicating number of fESC-specific genes that overlap with DMRs (ntESC, n=5; B-iPSC, n=18; F-iPSC, n=114). FIG. 13B is a heat map with grey bars in the right three lanes indicating number of fESC-specific genes that overlap with DMRs (ntESC, n=12; NP-iPSC, n=16; Bl-iPSC, n=45).

FIG. 14 is a series of pictorial and graphical representations showing DNA demethylation of promoters and gene expression on the selected pluripotent gene loci. FIG. 14A shows Oct4 (promoter regions corresponding to SEQ ID NOs:84-86 from left to right). FIG. 14B shows Nanog (promoter regions corresponding to SEQ ID NOs:87 and 88 from left to right). Schematic structure of the promoters are shown on top, and methylation status of the CpG sites measured by bisulfite pyrosequencing with three independent samples of fESC, ntESC, B-iPSC, and F-iPSC are shown in middle graphs. Detection of Oct4 and Nanog gene expression by RT-PCR with three independent samples of fESC, ntESC, B-iPSC, and F-iPSC are shown below each panel.

FIG. 15 is a series graphical representations of chimera analysis of fESC, ntESC, B-iPSC, and F-iPSC (refering to FIG. 6). FIG. 15A is of organ chimerism. B6CBA-derived cells were injected into blastocysts and transferred to pseudopregnant mice (N=3 clones of each stem cell type). Organs from E12.5 embryo (B-iPSC, n=14; F-iPSC, n=8; ntESC, n=15; fESC, n=13) were analyzed by flow cytometry to determine % GFP+ cells. Fibroblasts (MEF) were cultured in vitro for a week before analysis. The F-iPSC show poor contribution to not only fibroblasts but also to the entire spectrum of tissues, thus suggesting poor incorporation into the blastocyst. In vivo chimerism does not obviously reflect lineage bias, but also represents a very different assay from the in vitro analysis that is the focus of the paper. Error bars=s.d. FIG. 15B is germline transmission by flow cytometry analysis. Germ cells are represented by SSEA1+ cells of the embryonic gonad. fESC and B-iPSC don't contain GFP markers, but ntESC and F-iPSC harbor GFP markers. Donor cells were discriminated by GFP+ marker from either donor cells or blastocyst. SSEA1+ cells from donor cells were indicated in the box in the panels. Negative control: SSEA1 staining of heart cells from ntESC chimera mouse; Positive control: SSEA1 staining of gonad cells from GFP+ transgenic mouse.

FIG. 16 is a graphical representation of hematopoietic colony formation by fESC, NSC-NP-iPSC, and B-NP-iPSC. Average cell number per colony among 20 randomly picked colonies from fESC, NSC-NP-iPSC, and B-NP-iPSC are shown. Error bars=s.d.

FIG. 17 is a series of pictorial and graphical representations showing residual DNA methylation at hematopoiesis-related loci. FIG. 17A is of Gcnt2 gene. FIG. 17B is of Gata2 gene. Both genes show a greater degree of hypermethylation in Bl-iPSC relative to fESC compared to B-iPSC vs fESC. Upper panels show CHARM plots, while lower panels represent the degree of DNA methylation (of the CpG sites indicated in the box along x axis of the CpG density plot in the upper panels) as measured by bisulfite pyrosequencing.

FIG. 18 is a series of pictorial and graphical representations of analysis of DNA methylation at Wnt3. iPSCs that have higher hematopoietic potential (B-NPiPSC and NP-iPSC-TSA-AZA) show a greater degree of Wnt3 gene body methylation than the iPSCs that have lower hematopoietic potential (NSC-NPiPSC and NP-iPSC). Upper panel shows CHARM plots, while lower panel represents the degree of DNA methylation as measured by bisulfite pyrosequencing. The grey box indicated on the x axis of the CpG density plot in the upper panel marks the CpG sites that were measured by bisulfite pyrosequencing.

FIG. 19 is a series of graphical representations showing the relationship of Wnt3/3a on hematopoietic potential of NP-iPSC and NSC-NP-iPSC. FIG. 19A is a plot showing RNA levels from EBs differentiated for 3 days were harvested and analyzed by quantitative PCR, after normalization to (3-actin. Numbers represent fold expression of NP-iPSC-TSA-AZA (right bar of each set) relative to NP-iPSC (left bar of each set). FIG. 19B depicts methylcellulose analysis of blood-forming potential of iPSCs with Wnt3a treatment (+) between day 2-4 of EB differentiation compared to non-treated EBs (−). Error bars=s.d.

### DETAILED DESCRIPTION OF THE INVENTION

The present invention is based in part on the discovery that alterations in DNA methylation occur not only in promoters or CpG islands of an iPS cell genome during reprogramming of the cell, but in sequences up to 2 kb distant (termed “CpG island shores”). iPS cells are derived by epigenetic reprogramming, but their DNA methylation patterns have not previously been analyzed on a genome-wide scale. Substantial hypermethylation and hypomethylation of cytosine-phosphate-guanine (CpG) island shores in iPS cell lines as compared to ES cells and parental fibroblasts is described herein.

The DMRs in the reprogrammed cells (denoted R-DMRs) were significantly enriched in tissue-specific (T-DMRs) and cancer-specific DMRs (C-DMRs). Notably, even though iPS cells are derived from fibroblasts, their R-DMRs can distinguish between cells of normal tissue and between cancer and normal cells, e.g., colon cancer and normal colon cells. Thus, many DMRs are broadly involved in tissue differentiation, epigenetic reprogramming and cancer. Colocalization of hypomethylated R-DMRs with hypermethylated C-DMRs and bivalent chromatin marks, and colocalization of hypermethylated R-DMRs with hypomethylated C-DMRs and the absence of bivalent marks were observed, suggesting two mechanisms for epigenetic reprogramming in iPS cells and cancer.

The present invention is based in part on the discovery that induced pluripotent stem cells (iPSC) derived by factor-based reprogramming harbor residual DNA methylation signatures characteristic of their somatic tissue of origin, which favors their differentiation along lineages related to the donor cell, while restricting alternative cell fates. Somatic cell nuclear transfer and transcription factor-based reprogramming revert adult cells to an embryonic state, and yield pluripotent stem cells that can generate all tissues. These two reprogramming methods reset genomic methylation, an epigenetic modification of DNA that influences gene expression, by different mechanisms and kinetics. It was hypothesized that the resulting pluripotent stem cells might have different properties. The data presented herein show that low passage induced pluripotent stem cells (iPSC) derived by factor-based reprogramming harbor residual DNA methylation signatures characteristic of their somatic tissue of origin, which favors their differentiation along lineages related to the donor cell, while restricting alternative cell fates. Such an “epigenetic memory” of the donor tissue could be reset by differentiation and serial reprogramming, or by treatment of iPSC with chromatin-modifying drugs. In contrast, the differentiation and methylation of nuclear transfer-derived pluripotent stem cells were more similar to classical embryonic stem cells than were iPSC, consistent with more effective reprogramming Data herein demonstrate that factor-based reprogramming can leave an epigenetic memory of the tissue of origin that may influence efforts at directed differentiation for applications in disease modeling or treatment.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In accordance with this discovery, there are provided herein DMRs of reprogrammed iPS cells (R-DMRs) and methods of use thereof. In one aspect of the invention, there is provided a method of generating an iPS cell. The method includes contacting a somatic cell with an agent that alters the methylation status of one or more nucleic acid sequences of the somatic cell, the one or more nucleic acid sequences being outside of a promoter region of a gene and outside of a CpG island, and wherein the nucleic acid sequences are up to about 2 kb in distance from a CpG island, and wherein the nucleic acid sequences are differentially methylated in reprogrammed somatic cells as compared with parent somatic cells, thereby generating an iPS cell.

As used herein, reprogramming, is intended to refer to a process that alters or reverses the differentiation status of a somatic cell that is either partially or terminally differentiated. Reprogramming of a somatic cell may be a partial or complete reversion of the differentiation status of the somatic cell. In an exemplary aspect, reprogramming is complete wherein a somatic cell is reprogrammed into an iPS cell. However, reprogramming may be partial, such as reversion into any less differentiated state. For example, reverting a terminally differentiated cell into a cell of a less differentiated state, such as a multipotent cell.

As used herein, pluripotent cells include cells that have the potential to divide in vitro for an extended period of time (greater than one year) and have the unique ability to differentiate into cells derived from all three embryonic germ layers, namely endoderm, mesoderm and ectoderm.

Somatic cells for use with the present invention may be primary cells or immortalized cells. Such cells may be primary cells (non-immortalized cells), such as those freshly isolated from an animal, or may be derived from a cell line (immortalized cells). In an exemplary aspect, the somatic cells are mammalian cells, such as, for example, human cells or mouse cells. They may be obtained by well-known methods, from different organs, such as, but not limited to skin, brain, lung, pancreas, liver, spleen, stomach, intestine, heart, reproductive organs, bladder, kidney, urethra and other urinary organs, or generally from any organ or tissue containing living somatic cells, or from blood cells. Mammalian somatic cells useful in the present invention include, by way of example, adult stem cells, sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other known muscle cells, and generally any live somatic cells. In particular embodiments, fibroblasts are used. The term somatic cell, as used herein, is also intended to include adult stem cells. An adult stem cell is a cell that is capable of giving rise to all cell types of a particular tissue. Exemplary adult stem cells include hematopoietic stem cells, neural stem cells, and mesenchymal stem cells.

As discussed herein, alterations in methylation patterns occur during differentiation or dedifferention of a cell which work to regulate gene expression of critical factors that are ‘turned on’ or ‘turned off’ at various stages of differentiation. As such, one of skill in the art would appreciate that many types of agents are capable of altering the methylation status of one or more nucleic acid sequences of a somatic cell to induce pluripotency that may be suitable for use with the present invention.

An agent, as used herein, is intended to include any agent capable of altering the methylation status of one or more nucleic acid sequences of a somatic cell. For example, an agent useful in any of the method of the invention may be any type of molecule, for example, a polynucleotide, a peptide, a peptidomimetic, peptoids such as vinylogous peptoids, chemical compounds, such as organic molecules or small organic molecules, or the like. In various aspects, the agent may be a polynucleotide, such as DNA molecule, an antisense oligonucleotide or RNA molecule, such as microRNA, dsRNA, siRNA, stRNA, and shRNA.

MicroRNA (miRNA) are single-stranded RNA molecules whose expression is known to be regulated by methylation to play a key role in regulation of gene expression during differentiation and dedifferentiation of cells. Thus an agent may be one that inhibits or induces expression of miRNA or may be a mimic miRNA. As used herein, “mimic” microRNAs which are intended to mean a microRNA exogenously introduced into a cell that have the same or substantially the same function as their endogenous counterpart.

In various aspects of the present invention, an agent that alters the methylation status of one or more nucleic acid sequences is a nuclear reprogramming factor. Nuclear reprogramming factors may be genes that induce pluripotency and utilized to reprogram differentiated or semi-differentiated cells to a phenotype that is more primitive than that of the initial cell, such as the phenotype of a pluripotent stem cell. Those skilled in the art would understand that such genes and agents are capable of generating a pluripotent stem cell from a somatic cell upon expression of one or more such genes having been integrated into the genome of the somatic cell or upon contact of the somatic cell with the agent or expression product of the gene. As used herein, a gene that induces pluripotency is intended to refer to a gene that is associated with pluripotency and capable of generating a less differentiated cell, such as a pluripotent stem cell from a somatic cell upon integration and expression of the gene. The expression of a pluripotency gene is typically restricted to pluripotent stem cells, and is crucial for the functional identity of pluripotent stem cells.

Several genes have been found to be associated with pluripotency and suitable for use with the present invention as reprogramming factors. Such genes are known in the art and include, by way of example, SOX family genes (SOX1, SOX2, SOX3, SOX15, SOX18), KLF family genes (KLF1, KLF2, KLF4, KLF5), MYC family genes (C-MYC, L-MYC, N-MYC), SALL4, OCT4, NANOG, LIN28, STELLA, NOBOX, POU5F1 or a STAT family gene. STAT family members may include for example STAT1, STAT2, STAT3, STAT4, STAT5 (STAT5A and STAT5B), and STAT6. While in some instances, use of only one gene to induce pluripotency may be possible, in general, expression of more than one gene is required to induce pluripotency. For example, two, three, four or more genes may be simultaneously integrated into the somatic cell genome as a polycistronic construct to allow simultaneous expression of such genes. In an exemplary aspect, four genes are utilized to induce pluripotency including OCT4, POU5F1, SOX2, KLF4 and C-MYC. Additional genes known as reprogramming factors suitable for use with the present invention are disclosed in U.S. patent application Ser. No. 10/997,146 and U.S. patent application Ser. No. 12/289,873, incorporated herein by reference.

All of these genes commonly exist in mammals, including human, and thus homologues from any mammals may be used in the present invention, such as genes derived from mammals including, but not limited to mouse, rat, bovine, ovine, horse, and ape. Further, in addition to wild-type gene products, mutant gene products including substitution, insertion, and/or deletion of several (e.g., 1 to 10, 1 to 6, 1 to 4, 1 to 3, and 1 or 2) amino acids and having similar function to that of the wild-type gene products can also be used. Furthermore, the combinations of factors are not limited to the use of wild-type genes or gene products. For example, Myc chimeras or other Myc variants can be used instead of wild-type Myc.

The present invention is not limited to any particular combination of nuclear reprogramming factors. As discussed herein a nuclear reprogramming factor may comprise one or more gene products. The nuclear reprogramming factor may also comprise a combination of gene products as discussed herein. Each nuclear reprogramming factor may be used alone or in combination with other nuclear reprogramming factors as disclosed herein. Further, nuclear reprogramming factors of the present invention can be identified by screening methods, for example, as discussed in U.S. patent application Ser. No. 10/997,146, incorporated herein by reference. Additionally, the nuclear reprogramming factor of the present invention may contain one or more factors relating to differentiation, development, proliferation or the like and factors having other physiological activities, as well as other gene products which can function as a nuclear reprogramming factor.

The nuclear reprogramming factor may include a protein or peptide. The protein may be produced from a gene as discussed herein, or alternatively, in the form of a fusion gene product of the protein with another protein, peptide or the like. The protein or peptide may be a fluorescent protein and/or a fusion protein. For example, a fusion protein with green fluorescence protein (GFP) or a fusion gene product with a peptide such as a histidine tag can also be used. Further, by preparing and using a fusion protein with the TAT peptide derived from the virus HIV, intracellular uptake of the nuclear reprogramming factor through cell membranes can be promoted, thereby enabling induction of reprogramming only by adding the fusion protein to a medium thus avoiding complicated operations such as gene transduction. Since preparation methods of such fusion gene products are well known to those skilled in the art, skilled artisans can easily design and prepare an appropriate fusion gene product depending on the purpose.

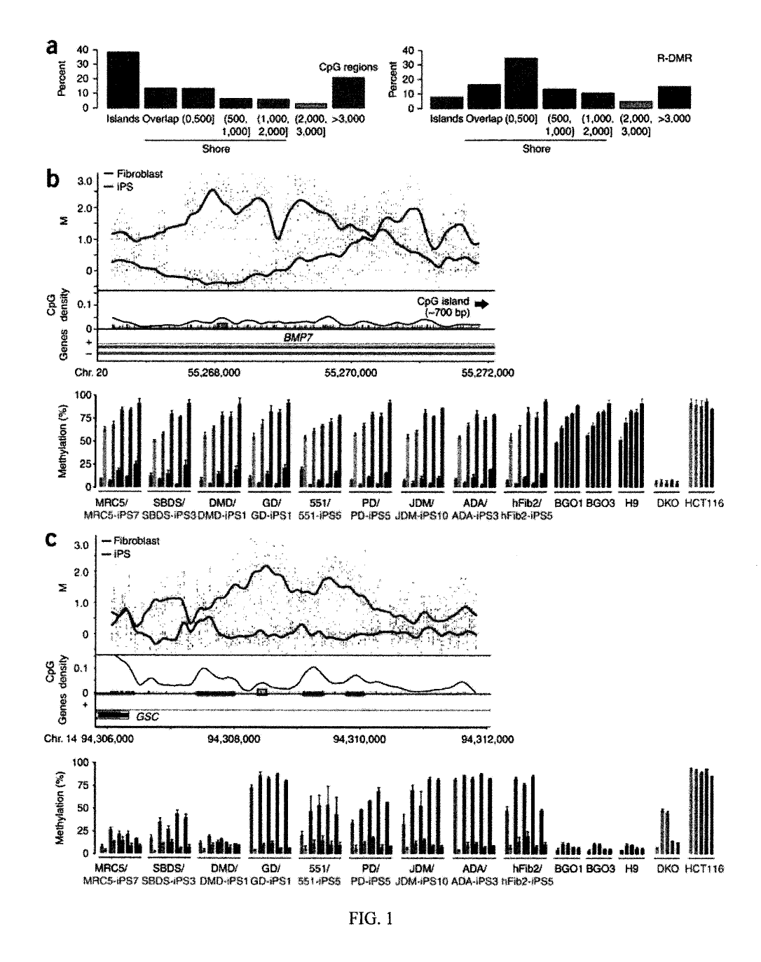

In certain embodiments, the agent alters the methylation status of one or more nucleic acid sequences, such as DMR sequences as set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof.

Detecting the methylation status profile of the one or more nucleic acid sequences of the induced iPS and/or comparing the methylation status profile to a methylation status profile of the one or more nucleic acid sequences of a parental somatic cell from which the iPS is induced may also be performed to assess pluripotency characteristics.

Similarly, expression profiling of reprogrammed somatic cells to assess their pluripotency characteristics may also be conducted. Expression of individual genes associated with pluripotency may also be examined. Additionally, expression of embryonic stem cell surface markers may be analyzed. As used herein, “expression” refers to the production of a material or substance as well as the level or amount of production of a material or substance. Thus, determining the expression of a specific marker refers to detecting either the relative or absolute amount of the marker that is expressed or simply detecting the presence or absence of the marker. As used herein, “marker” refers to any molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipd, a lipid, a lipoprotein or a small molecule.

Detection and analysis of a variety of genes known in the art to be associated with pluripotent stem cells may include analysis of genes such as, but not limited to OCT4, NANOG, SALL4, SSEA-1, SSEA-3, SSEA-4, TRA-1-60, TRA-1-81, or a combination thereof iPS cells may express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; γ-smooth muscle actin (γ-SMA); fibroblast growth factor 4 (Fgf4), Cripto, Daxl; zinc finger protein 296 (Zfp296); N-acetyltransferase-1 (Nati); ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utfl); Rexl; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; K1f4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tell); DPPA3/Stella; DPPA4; as well as other general markers for pluripotency, for example any genes used during induction to reprogram the cell. iPS cells can also be characterized by the down-regulation of markers characteristic of the differentiated cell from which the iPS cell is induced.

As used herein, “differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

“Differentiated cell” refers to a non-embryonic, non-parthenogenetic or non-pluripotent cell that possesses a particular differentiated, i.e., non-embryonic, state. The three earliest differentiated cell types are endoderm, mesoderm, and ectoderm.

Pluripotency can also be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types. Methods for producing chimeric animals are well known in the art and are described in U.S. Pat. No. 6,642,433, incorporated by reference herein.

Yet another method of confirming pluripotency is to observe cell differentiation into embryoid bodies and other differentiated cell types when cultured under conditions that favor differentiation (e.g., removal of fibroblast feeder layers).

The invention further provides iPS cells produced using the methods described herein, as well as populations of such cells. The reprogrammed cells of the present invention, capable of differentiation into a variety of cell types, have a variety of applications and therapeutic uses. The basic properties of stem cells, the capability to infinitely self-renew and the ability to differentiate into every cell type in the body make them ideal for therapeutic uses.

Accordingly, in one aspect the present invention further provides a method of treatment or prevention of a disorder and/or condition in a subject using induced pluripotent stem cells generated using the methods described herein. The method includes obtaining a somatic cell from a subject and reprogramming the somatic cell into an iPS cell using the methods described herein. The cell is then cultured under suitable conditions to differentiate the cell into a desired cell type suitable for treating the condition. The differentiated cell may then be introducing into the subject to treat or prevent the condition.

One advantage of the present invention is that it provides an essentially limitless supply of isogenic or synegenic human cells suitable for transplantation. The iPS cells are tailored specifically to the patient, avoiding immune rejection. Therefore, it will obviate the significant problem associated with current transplantation methods, such as, rejection of the transplanted tissue which may occur because of host versus graft or graft versus host rejection. Several kinds of iPS cells or fully differentiated somatic cells prepared from iPS cells from somatic cells derived from healthy humans can be stored in an iPS cell bank as a library of cells, and one kind or more kinds of the iPS cells in the library can be used for preparation of somatic cells, tissues, or organs that are free of rejection by a patient to be subjected to stem cell therapy.

The iPS cells of the present invention may be differentiated into a number of different cell types to treat a variety of disorders by methods known in the art. For example, iPS cells may be induced to differentiate into hematopoetic stem cells, muscle cells, cardiac muscle cells, liver cells, cartilage cells, epithelial cells, urinary tract cells, neuronal cells, and the like. The differentiated cells may then be transplanted back into the patient's body to prevent or treat a condition. Thus, the methods of the present invention may be used to treat a subject having a myocardial infarction, congestive heart failure, stroke, ischemia, peripheral vascular disease, alcoholic liver disease, cirrhosis, Parkinson's disease, Alzheimer's disease, diabetes, cancer, arthritis, wound healing, immunodeficiency, aplastic anemia, anemia, Huntington's disease, amyotrophic lateral sclerosis (ALS), lysosomal storage diseases, multiple sclerosis, spinal cord injuries, genetic disorders, and similar diseases, where an increase or replacement of a particular cell type/tissue or cellular de-differentiation is desirable.

In various embodiments, the method increases the number of cells of the tissue or organ by at least about 5%, 10%, 25%, 50%, 75% or more compared to a corresponding untreated control tissue or organ. In yet another embodiment, the method increases the biological activity of the tissue or organ by at least about 5%, 10%, 25%, 50%, 75% or more compared to a corresponding untreated control tissue or organ. In yet another embodiment, the method increases blood vessel formation in the tissue or organ by at least about 5%, 10%, 25%, 50%, 75% or more compared to a corresponding untreated control tissue or organ. In yet another embodiment, the cell is administered directly to a subject at a site where an increase in cell number is desired either before or after differentiation of the cell to a desired cell type.

Methylome analysis of iPS cells allows for the identification of such cells. As such, the present invention provides a method of identifying an iPS cell. The method includes comparing the methylation status of one or more nucleic acid sequences of a putative iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of an iPS cell, wherein a similarity in methylation status is indicative of the putative cell being an iPS cell. The known methylation status of the one or more nucleic acid sequences of an iPS cell may include the R-DMRs set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof.

Alternatively, the method of identifying an iPS cell includes comparing the methylation status of one or more nucleic acid sequences of a putative iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of a corresponding somatic cell from which the iPS cell is induced and/or an ES cell, wherein an alteration in methylation status is indicative of the putative cell being an iPS cell. As such, the one or more nucleic acid sequences may be DMR sequences as set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof.

The invention further provides a plurality of nucleic acid sequences, wherein the nucleic acid sequences are outside of a promoter region of a gene and outside of a CpG island, and wherein the nucleic acid sequence is up to about 2 kb in distance from a CpG island, and wherein the nucleic acid sequences are differentially methylated in the reprogramming of a somatic cell to generate an iPS cell. For example, the nucleic acid sequences are the DMR sequences as set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof.

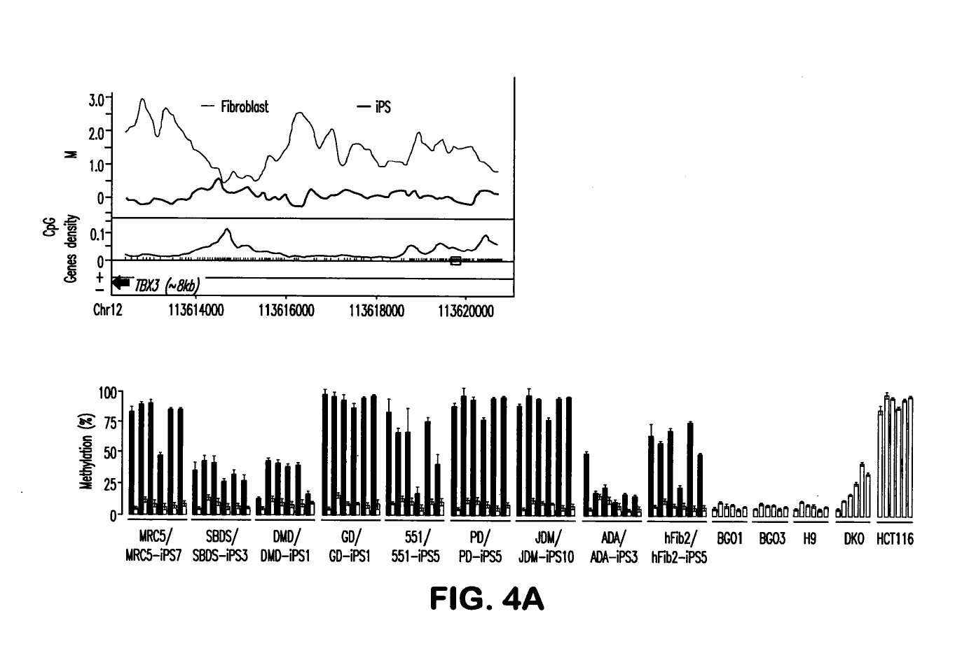

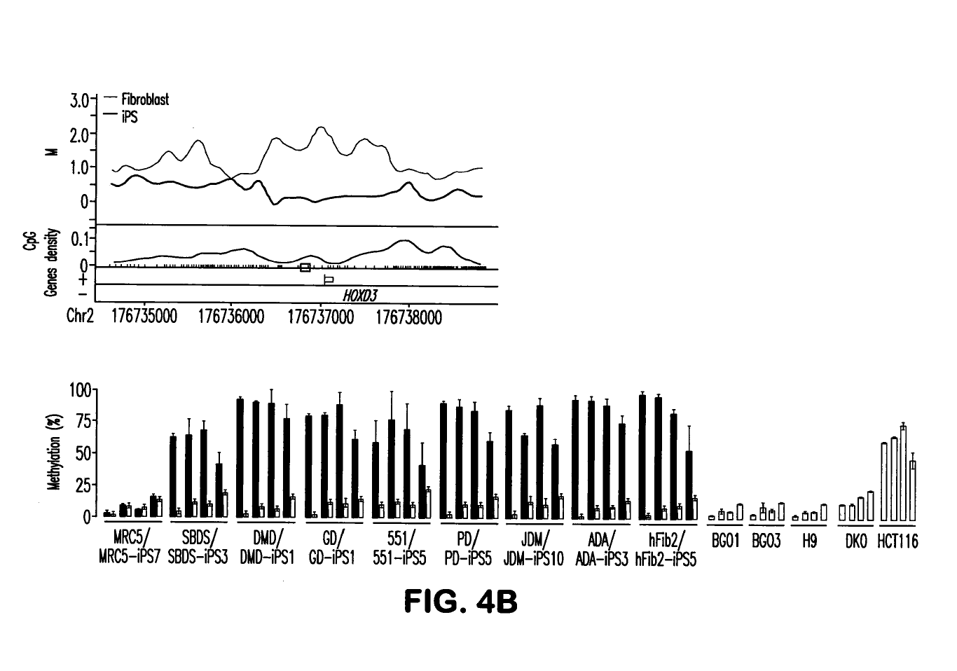

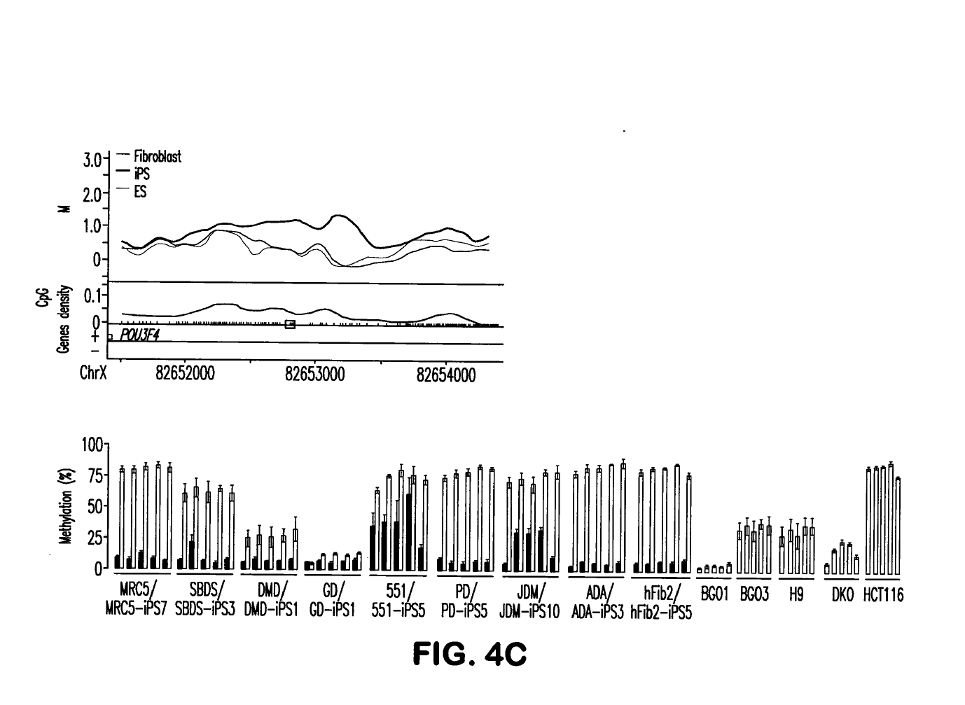

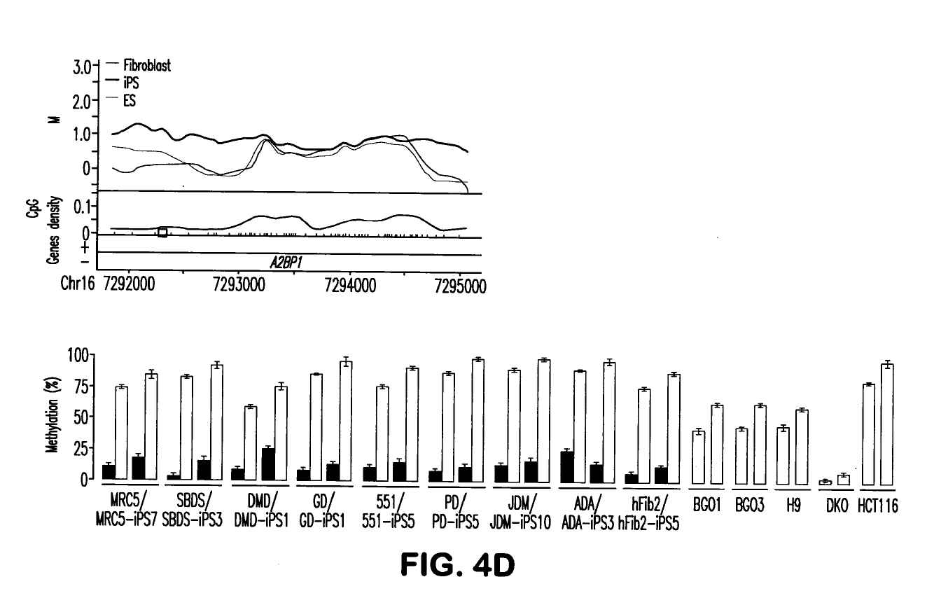

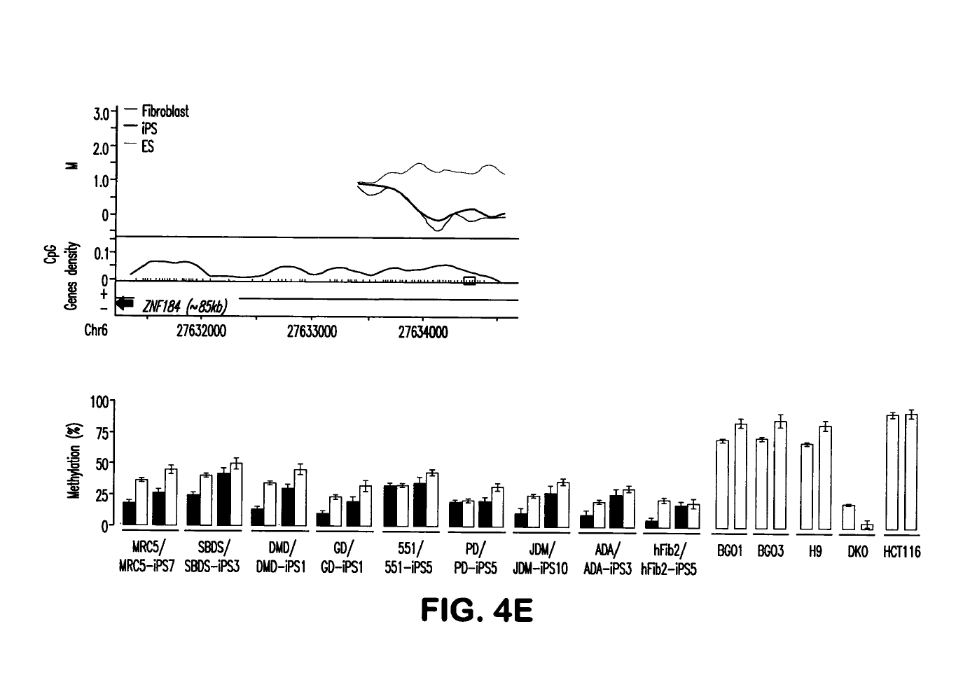

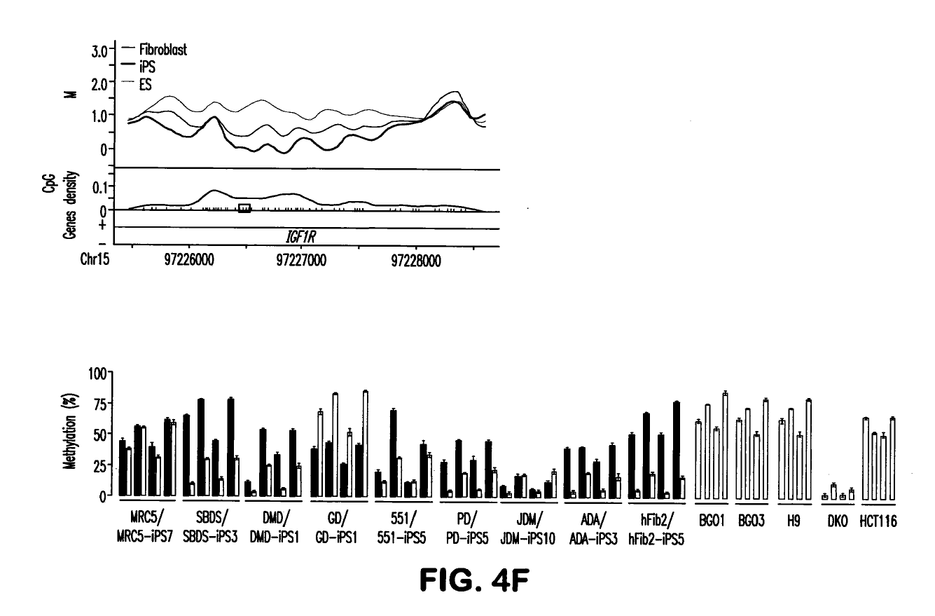

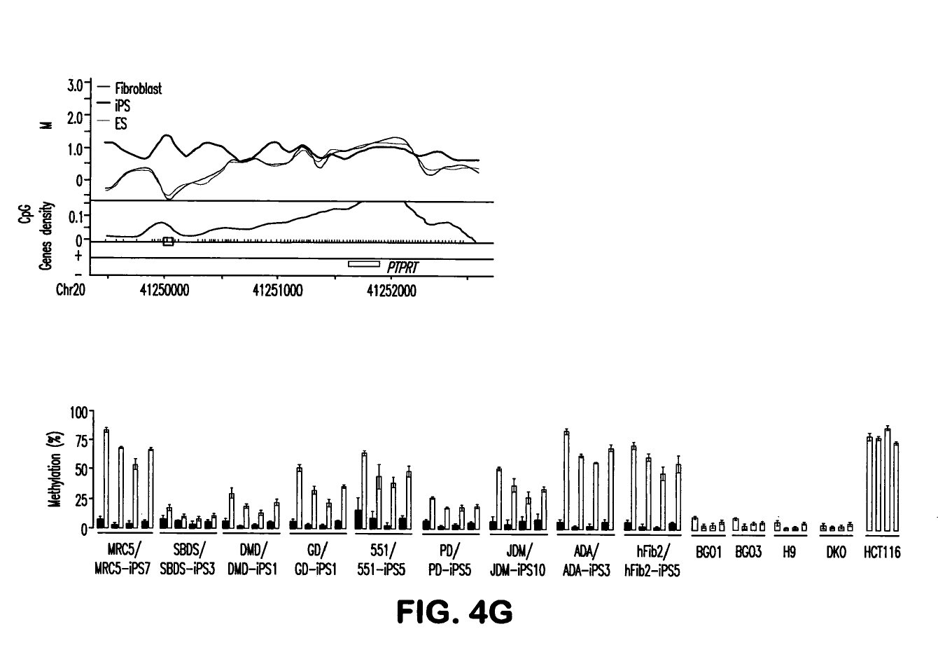

The invention further provides a plurality of nucleic acid sequences, wherein the nucleic acid sequences are outside of a promoter region of a gene and outside of a CpG island, and wherein the nucleic acid sequences are up to about 2 kb in distance from a CpG island, and wherein the methylation status of the nucleic acid sequences is altered in an iPS cell as compared to an ES cell. For example, the nucleic acid sequences are the DMR sequences as set forth in Table 7, FIGS. 4C-4G, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, or the C-MYC gene.

In various embodiments of the invention, the plurality of nucleic acid sequences may be utilized to provide a microarray for performing the methods described herein. One skilled in the art would appreciate the many techniques that are well known for attaching nucleic acids on a substrate that may be utilized along with the various types of substrates and configurations.

The invention further provides a method of characterizing the methylation status of the nucleic acid of an iPS cell. The method includes a) hybridizing labeled and digested nucleic acid of an iPS cell to a DNA microarray comprising at least 2000 nucleic acid sequences, with the proviso that the nucleic acid sequences are outside of a promoter region of a gene and outside of a CpG island, and wherein the nucleic acid sequences are up to about 2 kb in distance from a CpG island; and b) determining a pattern of methylation from the hybridizing of (a), thereby characterizing the methylation status for the iPS cell. In various embodiments, the one or more nucleic acid sequences are DMR sequences as set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof.

Characterizing the methylation status of the nucleic acid of an iPS cell may further include comparing the methylation status profile to a methylation profile from hybridization of the microarray with labeled and digested nucleic acid from a parental somatic cell from which the iPS is induced or from an ES cell. In particular embodiments, the one or more nucleic acid sequences are DMR sequences as set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof.

In various aspects of the invention, methylation status is converted to an M value. As used herein an M value, can be a log ratio of intensities from total (Cy3) and McrBC-fractionated DNA (Cy5): positive and negative M values are quantitatively associated with methylated and unmethylated sites, respectively.

In various aspects of the invention DMR may be hypermethylated or hypomethylated. Hypomethylation of a DMR is present when there is a measurable decrease in methylation of the DMR. In some embodiments, a DMR can be determined to be hypomethylated when less than 50% of the methylation sites analyzed are not methylated. Hypermethylation of a DMR is present when there is a measurable increase in methylation of the DMR. In some embodiments, a DMR can be determined to be hypermethylated when more than 50% of the methylation sites analyzed are methylated. Methods for determining methylation states are provided herein and are known in the art. In some embodiments methylation status is converted to an M value. As used herein an M value, can be a log ratio of intensities from total (Cy3) and McrBC-fractionated DNA (Cy5): positive and negative M values are quantitatively associated with methylated and unmethylated sites, respectively. M values are calculated as described in the Examples. In some embodiments, M values which range from −0.5 to 0.5 represent unmethylated sites as defined by the control probes, and values from 0.5 to 1.5 represent baseline levels of methylation.

Numerous methods for analyzing methylation status of a gene are known in the art and can be used in the methods of the present invention to identify either hypomethylation or hypermethylation of the one or more DMRs. In various embodiments, the determining of methylation status in the methods of the invention is performed by one or more techniques selected from the group consisting of a nucleic acid amplification, polymerase chain reaction (PCR), methylation specific PCR, bisulfite pyrosequenceing, single-strand conformation polymorphism (SSCP) analysis, restriction analysis, microarray technology, and proteomics. As illustrated in the Examples herein, analysis of methylation can be performed by bisulfite genomic sequencing. Bisulfite treatment modifies DNA converting unmethylated, but not methylated, cytosines to uracil. Bisulfite treatment can be carried out using the METHYLEASY bisulfite modification kit (Human Genetic Signatures).

In some embodiments, bisulfite pyrosequencing, which is a sequencing-based analysis of DNA methylation that quantitatively measures multiple, consecutive CpG sites individually with high accuracy and reproducibility, may be used. Exemplary primers for such analysis are set forth in Tables 11 and 12.

It will be recognized that depending on the site bound by the primer and the direction of extension from a primer, that the primers listed above can be used in different pairs. Furthermore, it will be recognized that additional primers can be identified within the DMRs, especially primers that allow analysis of the same methylation sites as those analyzed with primers that correspond to the primers disclosed herein.

Altered methylation can be identified by identifying a detectable difference in methylation. For example, hypomethylation can be determined by identifying whether after bisulfite treatment a uracil or a cytosine is present a particular location. If uracil is present after bisulfite treatment, then the residue is unmethylated. Hypomethylation is present when there is a measurable decrease in methylation.

In an alternative embodiment, the method for analyzing methylation of the DMR can include amplification using a primer pair specific for methylated residues within a DMR. In these embodiments, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

Other methods are known in the art for determining methylation status of a DMR, including, but not limited to, array-based methylation analysis and Southern blot analysis.

Methods using an amplification reaction, for example methods above for detecting hypomethylation or hypermethylation of one or more DMRs, can utilize a real-time detection amplification procedure. For example, the method can utilize molecular beacon technology (Tyagi et al., Nature Biotechnology, 14: 303 (1996)) or Taqman™ technology (Holland et al., Proc. Natl. Acad. Sci. USA, 88:7276 (1991)).

Also methyl light (Trinh et al., Methods 25(4):456-62 (2001), incorporated herein in its entirety by reference), Methyl Heavy (Epigenomics, Berlin, Germany), or SNuPE (single nucleotide primer extension) (see e.g., Watson et al., Genet Res. 75(3):269-74 (2000)) Can be used in the methods of the present invention related to identifying altered methylation of DMRs.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, for example, whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, e.g., Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

The degree of methylation in the DNA associated with the DMRs being assessed, may be measured by fluorescent in situ hybridization (FISH) by means of probes which identify and differentiate between genomic DNAs, associated with the DMRs being assessed, which exhibit different degrees of DNA methylation. FISH is described, for example, in de Capoa et al. (Cytometry. 31:85-92, 1998) which is incorporated herein by reference. In this case, the biological sample will typically be any which contains sufficient whole cells or nuclei to perform short term culture. Usually, the sample will be a sample that contains 10 to 10,000, or, for example, 100 to 10,000, whole cells.

Additionally, as mentioned above, methyl light, methyl heavy, and array-based methylation analysis can be performed, by using bisulfate treated DNA that is then PCR-amplified, against microarrays of oligonucleotide target sequences with the various forms corresponding to unmethylated and methylated DNA.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In another aspect, the present invention includes kits that are useful for carrying out the methods of the present invention. The components contained in the kit depend on a number of factors, including: the particular analytical technique used to detect methylation or measure the degree of methylation or a change in methylation, and the one or more DMRs is being assayed for methylation status.

Accordingly, the present invention provides a kit for determining a methylation status of one or more DMRs of the invention. In some embodiments, the one or more DMRs are selected from one or more of the sequences as set forth in Tables 2, 6, 9, 12, 14, 15, FIGS. 1B-1C, FIGS. 4A-4G, 10, 14, 17, 18, the BMP7 gene, the GSC gene, the TBX3 gene, the HOXD3 gene, the PTPRT gene, the POU3F4 gene, the AZBP1 gene, the ZNF184 gene, the IGF1R gene, the POU5F1 gene, the NANOG gene, the OCT4 gene, the SOX2 gene, the KLF4 gene, the C-MYC gene and any combination thereof. The kit includes an oligonucleotide probe, primer, or primer pair, or combination thereof for carrying out a method for detecting hypomethylation, as discussed above. For example, the probe, primer, or primer pair, can be capable of selectively hybridizing to the DMR either with or without prior bisulfite treatment of the DMR. The kit can further include one or more detectable labels.

The kit can also include a plurality of oligonucleotide probes, primers, or primer pairs, or combinations thereof, capable of selectively hybridizing to the DMR with or without prior bisulfite treatment of the DMR. The kit can include an oligonucleotide primer pair that hybridizes under stringent conditions to all or a portion of the DMR only after bisulfite treatment. In one aspect, the kit can provide reagents for bisulfite pyrosequencing including one or more primer pairs set forth in Tables 11 and 12. The kit can include instructions on using kit components to identify, for example, the presence of cancer or an increased risk of developing cancer.

To examine DNAm on a genome-wide scale, comprehensive high-throughput array-based relative methylation (CHARM) analysis, which is a microarray-based method agnostic to preconceptions about DNAm, including location relative to genes and CpG content was carried out. The resulting quantitative measurements of DNAm, denoted with M, are log ratios of intensities from total (Cy3) and McrBC-fractionated DNA (Cy5): positive and negative M values are quantitatively associated with methylated and unmethylated sites, respectively. For each sample, ˜4.6 million CpG sites across the genome of iPS cells, parental somatic cells and ES cells were analyzed using a custom-designed NimbleGen HD2 microarray, including all of the classically defined CpG islands as well as all nonrepetitive lower CpG density genomic regions of the genome. 4,500 control probes were included to standardize these M values so that unmethylated regions were associated, on average, with values of 0. CHARM is 100% specific at 90% sensitivity for known methylation marks identified by other methods (for example, in promoters) and includes the approximately half of the genome not identified by conventional region preselection. The CHARM results were also extensively corroborated by quantitative bisulfite pyrosequencing analysis.

Provided herein is a genome-wide analysis of DNA methylation addressing variation among iPS cells, somatic cells and ES cells, revealing several surprising differences and relationships among the cell types of epigenetic variation, supported by extensive bisulfite pyrosequencing and functional analysis. First, most cell-specific DNAm was found to occur, not at CpG islands, but at CpG island shores (sequences up to 2 kb distant from CpG islands). The identification of these regions opens the door to functional studies, such as those investigating the mechanism of targeting DNAm to these regions and the role of differential methylation of shores.

IPS cells are derived by epigenetic reprogramming, but their DNA methylation patterns have not previously been analyzed on a genome-wide scale. Substantial hypermethylation and hypomethylation of cytosine-phosphate-guanine (CpG) island shores in nine human iPS cell lines as compared to their parental fibroblasts was determined. The R-DMRs in the reprogrammed cells were significantly enriched in tissue-specific (T-DMRs; 2.6-fold, P<10⁻⁴) and cancer-specific DMRs (C-DMRs; 3.6-fold, P<10⁻⁴). Notably, even though the iPS cells are derived from fibroblasts, their R-DMRs can distinguish between normal brain, liver and spleen cells and between colon cancer and normal colon cells. Thus, many DMRs are broadly involved in tissue differentiation, epigenetic reprogramming and cancer. Colocalization of hypomethylated R-DMRs with hypermethylated C-DMRs and bivalent chromatin marks, and colocalization of hypermethylated R-DMRs with hypomethylated C-DMRs and the absence of bivalent marks were observed, suggesting two mechanisms for epigenetic reprogramming in iPS cells and cancer.

In one aspect of the invention, methylation density is determined for a region of nucleic acid. Density may be used as an indication of production of an iPS cell, for example. A density of about 0.2 to 0.7, about 0.3 to 0.7, 0.3 to 0.6 or 0.3 to 0.4, or 0.3, may be indicative of generation of an iPS cell (the calculated DNA methylation density is the number of methylated CpGs divided by the total number of CpGs sequenced for each sample). Methods for determining methylation density are well known in the art. For example, a method for determining methylation density of target CpG islands has been established by Luo et al. Analytical Biochemistry, Vol. 387:2 2009, pp. 143-149. In the method, DNA microarray was prepared by spotting a set of PCR products amplified from bisulfite-converted sample DNAs. This method not only allows the quantitative analysis of regional methylation density of a set of given genes but also could provide information of methylation density for a large amount of clinical samples as well as use in the methods of the invention regarding iPS cell generation and detection. Other methods are well known in the art (e.g., Holemon et al., BioTechniques, 43:5, 2007, pp. 683-693).

The following examples are provided to further illustrate the advantages and features of the present invention, but are not intended to limit the scope of the invention. While they are typical of those that might be used, other procedures, methodologies, or techniques known to those skilled in the art may alternatively be used.

### Example I

### Differential Methylation of Tissue and Cancer Specific CpG Island Shores Distinguishes Human iPS Cells, ES Cells and Fibroblasts

The following experimental protocols and materials were utilized.

Summary: ES cells and iPSCs were cultured in ESC media containing 15% FBS, and 1,000 U/ml of LIF. For the reprogramming of somatic cells, retrovirus expressing Oct4, Sox2, K1f4, and Myc were introduced. For the somatic cells containing inducible reprogramming factors, the media was supplemented with 2 ng/ml of doxycycline. For DNA and RNA isolation, fESC or iPSCs were trypsinized and re-plated onto new tissue culture dishes for 45 minutes to remove feeder cells, and nucleic acids were extracted from the non-adherent cell suspension. Genomic DNA methylation analysis and pyrosequencing were performed by previously published methods.

Cell culture and isolation of RNA and genomic DNA from fibroblast, hES cells and iPS cells. iPS cell lines and their parental fibroblasts used were described in Park et al. (Nature (451)141-146 (2008)) and Park et al. Cell (134)877-886 (2008)).

MRC5 (14-week-old fetal lung fibroblast from the ATCC cell biology collection), Detroit 551 (551, fetal skin fibroblast from ATCC), hFib2 (adult dermal fibroblast), SBDS (DF250), DMD (GM04981 from Coriell), GD (GM00852A from Coriell), PD (AG20446 from Coriell), JDM (GMO2416 from Coriell) and ADA (GM01390 from Coriell). Human ES cells BGO1, BGO3 and H9 were used. Fibroblasts were grown in a-MEM containing 10% inactivated fetal serum, 50 U/ml penicillin, 50 mg/ml streptomycin and 1 mM L-glutamine. hES cells and iPS cells were cultured in hES medium (80% DMEM/F12, 20% KO Serum Replacement™, 10 ng/ml bFGF, 1 mM L-glutamine, 100 nM nonessential amino acids, 100 nM 2-mercaptoethanol, 50 U/ml penicillin and 50 mg/ml streptomycin). Total RNA and genomic DNA were isolated using RNeasy™ kit (Qiagen) with in-column DNase treatment and DNeasy™ kit (Qiagen), respectively, according to manufacturer's protocol.

CHARM DNA methylation analysis. For each sample, 5 ng of genomic DNA was digested, fractionated, labeled and hybridized to a CHARM microarray as described in Irizarry et al. (Nat. Genet. (41)178-186 (2009)) and Irizarry et al. (Genome Res. (18)780-790 (2008)).

CHARM microarrays were prepared using custom-designed NimbleGen™ HD2 microarrays as described in Irizarry et al. (Nat. Genet. (41)178-186 (2009)) and Irizarry et al. (Genome Res. (18)780-790 (2008)). For each probe, the averaged methylation (M) values across the same cell type were computed and were used to find regions of differential methylation (ΔM) for each pairwise cell type comparison. The absolute area of each region was calculated by multiplying the number of probes by ΔM. For the first experiment (n=6 for each cell type), false discovery rates (FDR) were computed and a cutoff of 5% was used to define the R-DMRs. The parental fibroblast lines for this experiment were MRCS, SBDS, DMD, GD, Detroit551 and PD, and they were compared to the iPS cell lines derived from them. For the second set of experiments (n=3 for each cell type), an absolute FDR could not be calculated, so an absolute area cutoff of 10.0 was used, which corresponded in magnitude to the 5% FDR cutoff of the first set of experiments. The parental fibroblast lines for the second experiment were JMD, ADA and hFib2 and were compared to the iPS cell lines derived from them, as well as to three ES cell lines, BGO1, BGO3 and H9.

Overlap of R-DMRs with bivalent domains, transcription factor (POU5F1, NANOG, SOX2) binding sites, T-DMRs and C-DMRs. The number of overlapping regions for hypermethylated R-DMRs and hypomethylated R-DMRs were computed for overlaps with bivalent domains as in Bernstein et al. (Cell (125)315-326 (2006)) and Pan et al. (Cell Stem Cell (1)299-312 (2007)). The number of overlapping regions for hypermethylated R-DMRs and hypomethylated R-DMRs were computed for overlaps with POU5F1, NANOG and SOX2 binding sites as described in Boyer et al. (Cell (122)947-956 (2005)). The number of overlapping regions for hypermethylated R-DMRs and hypomethylated R-DMRs were computed for overlaps with tissue-specific differentially methylated regions (T-DMRs) as described in Irizarry et al. (Nat. Genet. (41)178-186 (2009)). The number of overlapping regions for hypermethylated R-DMRs and hypomethylated R-DMRs were computed for overlaps with cancer-specific differentially methylated regions (C-DMRs) as described in Irizarry et al. (Nat. Genet. (41)178-186 (2009)). To determine the significance of each overlap, randomly generated CHARM array regions equal to the number and lengths of the R-DMRs were generated and P values were calculated by 10,000 permutations. Random values were calculated as the average over all 10,000 permutations.

Unsupervised cluster analysis. Using the R-DMRs, unsupervised cluster analysis was performed to determine to what degree the methylation at these locations distinguished normal brain, liver and spleen as well as colon cancer from its matched counterpart. As a test of significance, 1,000 CHARM array regions of length and number equal to those of the R-DMRs were randomly generated and then assessed the median euclidean distance among samples of a given tissue type and the median euclidean distance among samples of different tissue types. This test was also applied to the cancer and normal samples.

Bisulfite pyrosequencing. For validation of DMRs, 500 ng of genomic DNA from each sample was treated with bisulfite using an EpiTect Kit (Qiagen) according to the manufacturer's specifications. Bisulfite-treated genomic DNA was PCR-amplified using unbiased nested primers and performed quantitative pyrosequencing using a PSQ HS96 (Biotage). The DNA methylation percentage at each CpG site was determined using the Q-CpG methylation software (Biotage). Control DNA was from the generally highly methylated HCT116 colon cancer cell line, as well as from a hypomethylated double DNA methyltransferase 1/3 B knockout somatic cell line derived from it. Table 11 provides the primer sequences used for the bisulfite pyrosequencing reactions, as well as the chromosomal coordinates in the University of California at Santa Cruz March 2006 human genome assembly for each CpG site interrogated. The annealing temperature used for all PCR reactions was 50° C.

Affymetrix microarray expression analysis. Genome-wide gene expression analysis was done using Affymetrix U133 Plus 2.0™ microarrays. For each sample, 1 μg of high-quality total RNA was amplified, labeled and hybridized onto the microarray according to Affymetrix's specifications, and data were normalized as previously described in Irizarry et al. (Biostatistics (4)249-264 (2003)).

GO annotation. GO annotation was performed as described in Dennis et al. (Genome Biol. 4, R60 (2003)) and Huang et al. (Nat. Protocols (4)44-57 (2009)).

Accession codes. NCBI GEO: Gene expression microarray data and CHARM microarray data have been submitted under accession number GSE18111.

URLs. A complete set of R-DMRs can be found at rafalab.jhsph.edu/r-dmrplots.pdf. A complete set of ES-iPS DMRs can be found at rafalab.jhsph.edu/es-ipsdmr pdf.

IPS cells can be derived from somatic cells by introduction of a small number of genes: for example, POU5F1, MYC, KLF4 and SOX2. As direct derivatives of an individual's own tissue, iPS cells offer considerable therapeutic promise, avoiding both immunologic and ethical barriers to their use. iPS cells differ from their somatic parental cells epigenetically, and thus a comprehensive comparison of the epigenome in iPS and somatic cells would provide insight into the mechanism of tissue reprogramming. Although two previous targeted studies examined a subset of the genome, 7,000 (Ball et al. (Nat. Biotechnol. (27)485 (2009)) and 66,000 (Deng et al. (Nat. Biotechnol. (27)353-360 (2009))) CpG sites in a small cohort of three iPS-fibroblast pairs, a global assessment of genome-wide methylation has not yet been performed.

Recently, differential methylation patterns that distinguish among normal tissue types (T-DMRs) and patterns that can segregate colorectal cancer tissue from matched normal tissues (C-DMRs) were described. Unexpectedly, these two DMRs occur 13-fold more frequently at CpG island ‘shores’, regions of comparatively low CpG density that are located near traditional CpG islands, than at the CpG islands themselves. Cancers showed approximately equal numbers of hypomethylated and hypermethylated regions, and 45% of C-DMRs overlapped T-DMRs, suggesting that epigenetic changes in cancer involve reprogramming of the normal pattern of tissue-specific differentiation.

Here differential methylation patterns in iPS cell reprogramming was explored, first comparing six human iPS cell lines to the fibroblasts from which they were derived using comprehensive high-throughput array-based relative methylation (CHARM) analysis as described in Irizarry et al. (Genome Res. (18)780-790 (2008)). This approach allows the interrogation of ˜4.6 million CpG sites genome-wide using a custom designed NimbleGen™ HD2 microarray, including almost all CpG islands and shores in the human genome. Genomic DNA from iPS cells, their parental fibroblasts and human embryonic stem (hES) cells was digested with the enzyme McrBC, fractionated, labeled and hybridized to a CHARM array.

A total of 4,401 regions (including 96,404 CpG sites) were found to differ in iPS cell lines from the fibroblasts of origin (Tables 1 and 2) at a false discovery rate (FDR) of 5%; these regions were termed R-DMRs. Of these R-DMRs, DMRs that were hypermethylated in iPS cells compared to fibroblasts predominated over hypomethylated DMRs (60%:40%). Of the 4,401 DMRs, 1,969 were within 2 kb of the transcriptional start site of a gene.

The genes that were associated with these R-DMRs showed functionally important features based on bioinformatic analyses. First, gene ontology (GO) annotation analysis of these genes revealed significant enrichment for genes involved in developmental and regulatory processes (Table 3). For example, 38% of the genes that were hypomethylated in iPS compared to fibroblasts (P=3.56×10⁻⁶⁰) and 22% of the genes that were hypermethylated in iPS compared to fibroblasts (P=1.73×10⁻¹²) were involved in developmental processes. To further elucidate the functional significance of these R-DMRs, their overlap with bivalent domains, which mark developmental genes in ES cells was examined Notably, 65% of the R-DMRs that were hypomethylated in iPS cells compared to fibroblasts showed significant association with bivalent domain marks (P<0.0001 by 10,000 permutations), whereas only 18.6% of hypermethylated R-DMRs overlapped with these domains (P=0.5699 by 10,000 permutations) (Table 4). Furthermore, when the overlap of the R-DMRs was observed with known binding sites for pluripotency markers such as POU5F1, NANOG and SOX2 as discussed in Boyer et al. (Cell (122)947-956 (2005)), a similar relationship was seen, in which the hypomethylated R-DMRs showed significant overlap (P<0.0001 by 10,000 permutations) whereas the hypermethylated DMRs did not (P=1 by 10,000 permutations; Table 5). These observations indicate that the sites of demethylation during reprogramming of fibroblasts to iPS cells are tightly linked to genes that are functionally important for pluripotency.

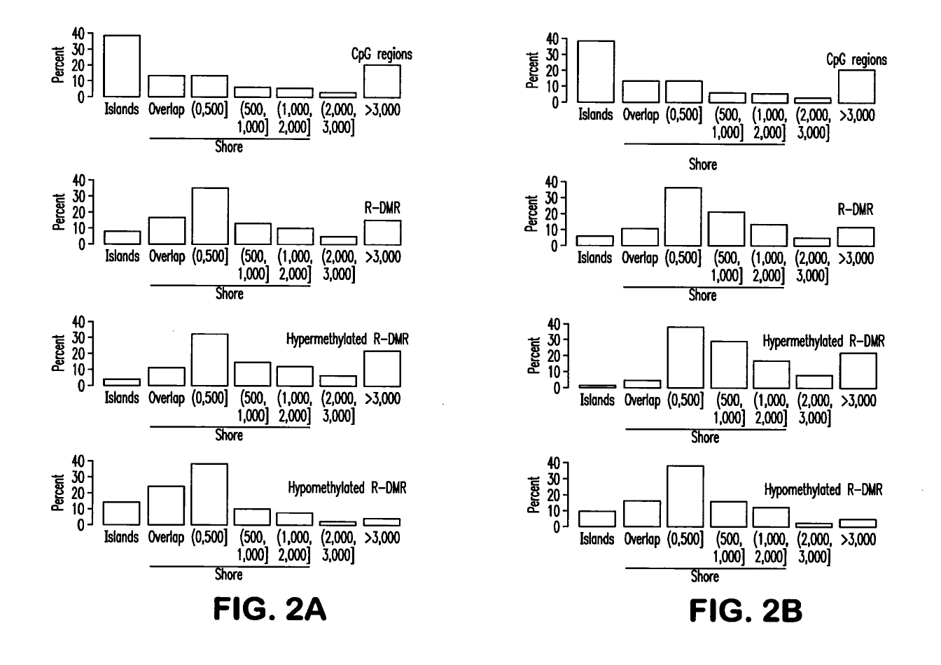

The R-DMRs showed several noteworthy features. First, over 70% of the R-DMRs were associated with CpG island shores rather than with the associated CpG islands (FIG. 1A), regardless of whether the R-DMRs were hypermethylated or hypomethylated in iPS cells relative to fibroblasts (FIG. 2A). Second, 56% of R-DMRs overlapped T-DMRs previously identified as distinguishing tissues representing the three germ cell lineages, namely, brain, liver and spleen (Table 1). This overlap was statistically significant (P<0.0001 by 10,000 permutations). Furthermore, both hypermethylated and hypomethylated R-DMRs in iPS cells showed similar overlap with known T-DMRs, overlapping at 54% and 60%, respectively (Table 1). Thus, R-DMRs are heavily enriched in CpG island shores and largely overlap T-DMRs that are involved in normal development. There was also a 61% overlap of the gene-proximal R-DMRs with the T-DMRs.

FIG. 1 details reprogramming differentially methylated regions (R-DMRs). FIG. 1A depicts enrichment of R-DMRs at CpG island shores. The CHARM array (left, labeled CpG regions) is enriched in CpG islands, and the R-DMRs (right, labeled R-DMR) show marked enrichment at CpG island shores. Islands are denoted as regions that include >50% of a CpG island or are wholly contained in an island, and overlap regions are denoted as regions that include 0.1-50% of a CpG island. Specific base intervals of regions not overlapping islands are indicated; (0-500) means from 1 to 500 bases. Percentage of the distribution (y axis) is given for the CpG regions (CHARM array, null hypothesis) and reprogramming differentially methylated regions (R-DMRs). FIGS. 1B and C show examples of DMRs. The gene encoding bone morphogenetic protein 7 (BMP7) is indicated in B, and the gene encoding goosecoid (GSC) is indicated in C. In each case, the upper panels show a plot of methylation (M value; see Methods) versus genomic location, where the curve represents averaged smoothed M values; the location of CpG dinucleotides (black tick marks), CpG density, location of CpG islands (filled boxes along the x-axis (zero)), as well as the gene annotation are shown. The bottom panels show validation by bisulfite pyrosequencing (mapping to unfilled box in upper panel). Bars represent the mean methylation (triplicate measurement)±s.d. of iPS cells, fibroblasts and ES cells (BGO1, BGO3 and H9) as well as the generally highly methylated HCT116 colon cancer cell line and a generally hypomethylated double DNA methyltransferase 1/3B double knockout line (DKO) derived from it. In each case, five separate CpG sites were assayed quantitatively, shown as differing shades.

FIG. 2 depicts the distribution of distance of reprogramming differentially methylated regions (R-DMRs) from CpG islands. Islands are regions that are inside, cover, or overlap more than 50% of a CpG island. Overlap are regions that overlap 0.1-50% of a CpG island. Regions denoted by (0, 500] are regions located ≦500 bp but do not overlap an island. Regions denoted by (500, 1000] are regions located >500 bp and ≦1000 bp from an island. Regions denoted by (1000, 2000] are regions located >1000 bp and ≦2000 bp from an island. Regions denoted by (2000, 3000] are regions located >2000 bp and ≦3000 bp from an island. Regions denoted by >3000 are >3000 bp from an island. Percentage are given for the CpG regions (CHARM array, null hypothesis) and reprogramming differentially methylated regions (R-DMRs) as well as the R-DMRs subdivided into hypermethylation and hypomethylation in iPS relative to fibroblast. Percentages of each class is given for (A) R-DMRs from the first experiment (n=6 for each cell type) (R-DMR panel is duplicated from FIG. 1A) and (B) R-DMRs from second experiment (n=3 for each cell type).

The CHARM analysis was then repeated on a separate set of three iPS cell lines and the fibroblasts from which they were derived, as well as three human ES cell lines. It was not possible to perform an FDR statistical test on this smaller number of lines, so a similar area cutoff in the curves was used that corresponded in magnitude to the 5% FDR cutoff of the previous experiment. In this second analysis, 2,179 R-DMRs were identified, with a slight excess of hypomethylated versus hypermethylated DMRs (55% compared to 45%) in iPS cells. Notably, 80% of the DMRs overlapped those found in the first experiment (see Table 6 for full list). As in the first analysis, there was a substantial enrichment for CpG island shores (78%, FIG. 2B), and 60% of the R-DMRs overlapped T-DMRs (Table 1).

This second analysis provided insight into the methylome of iPS cells as compared to ES cells. Although the two cell types had very similar DNA methylation, 71 DMRs distinguished them, with 51 showing hypermethylation and 20 showing hypomethylation in iPS cells (Table 7). GO annotation of these DMRs showed significant enrichment of developmental processes in the genes that were hypermethylated in iPS cells as compared to ES cells (Table 8). In 32 of the DMRs that distinguish iPS cells from ES cells, the DMRs were near genes of interest, including HOXA9 and two genes that encode the zinc finger proteins ZNF568 and ZFP112. In some cases, the methylation in iPS cells was intermediate between differentiated fibroblasts and ES cells; this was true, for example, of TBX5, which encodes a transcription factor that is involved in cardiac and limb development. In other cases, methylation in iPS cells differed from both fibroblasts and ES cells, suggesting that the iPS cells occupy a distinct and possibly aberrant epigenetic state. An example was PTPRT, encoding a protein tyrosine phosphatase involved in many cellular processes including differentiation. For some ES-iPS differences, the methylation levels changed in the same direction as for ES cells compared to fibroblasts, but to a greater degree; for example, methylation of the homeobox gene HOXA9 was greater in iPS compared to ES, whose methylation at this gene was greater than in fibroblasts.

These data were validated in two ways. First the methylation results from CHARM were verified by bisulfite pyrosequencing of nine DMRs, examining 2-6 CpGs within each DMR. For all of these genes, the bisulfite pyrosequencing data confirmed the differential methylation data from CHARM (FIGS. 1B and C; FIG. 4).

FIG. 4 includes examples of differential DNA methylation (upper panels) and confirmation by bisulfite pyrosequencing (lower panels). Upper panels are a plot of M value versus genomic location, where the curve represents averaged smoothed M values. Also shown in the upper panels are the locations of CpG dinucleotide (black tick marks on x axis), CpG density (smoothed black line) calculated across the region using a standard density estimator, location of CpG islands (filled boxes along the x-axis (zero)), as well as gene annotation indicating the transcript (thin outer gray line), coding region (thin inner gray line), exons (filled gray box) and gene transcription directionality on the y axis (sense marked as +, antisense as −). The lower panels represent the degree of DNA methylation as measured by bisulfite pyrosequencing. The unfilled box indicated on the x axis of the CpG density plot in the upper panel indicates the CpG sites that were measured. Reactions were done in triplicate; bars represent the mean methylation±SD of iPS cells, fibroblasts, and ES cells (BGO1, BGO3 and H9) as well as DKO (DNMT1 and DNMT3B Double KO cell line) and HCT116 (parental colon cancer cell line) for each individual CpG site measured. FIGS. A and B are DMRs found by comparison between iPS cells and fibroblast (n=6), (c-g) is a DMR found by comparison between iPS cells and ES cells (n=3). (A) TBX3 (T-box 3 protein), (B) HOXD3 (Homeobox D3), (C) POU3F4 (POU domain, class 3, transcription factor 4), (D) A2BP1 (ataxin 2-binding protein 1), (E) ZNF184 (zinc finger protein 184), (F) IGF1R (insulin-like growth factor 1 receptor), (G) PTPRT (protein tyrosine phosphatase, receptor type, T).

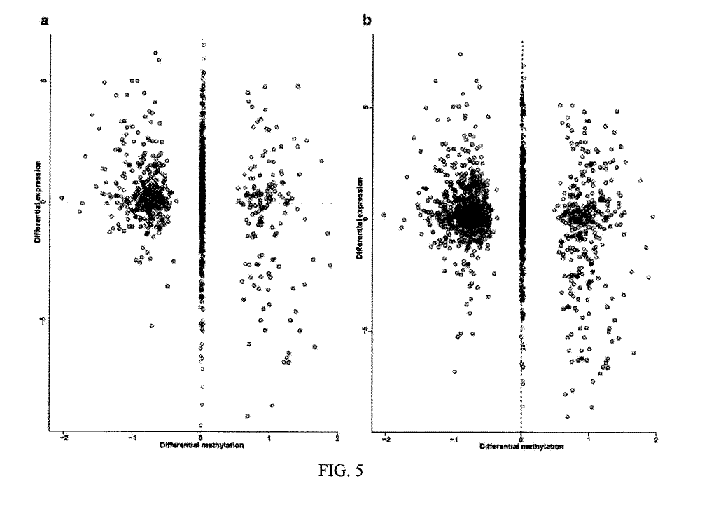

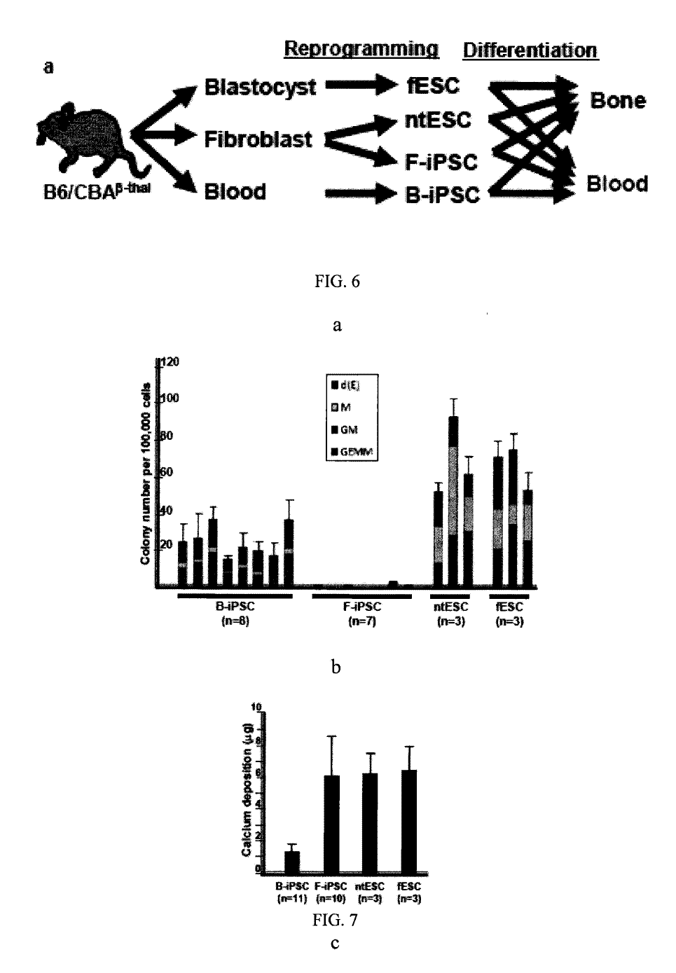

Global gene expression analysis was also performed using the Affymetrix HGU133 Plus™ 2.0 microarray. There was a strong inverse correlation between differential gene expression and differential DNA methylation at R-DMRs that are within 500 bp of the transcriptional start site (TSS) of a gene: P<10−3 for both hypermethylation and hypomethylation (FIG. 5, Table 9). The significant association held true even when the R-DMR was within 1 kb of a TSS (P=0.01 and P<10−3 for hypermethylated and hypomethylated R-DMRs, respectively, FIG. 5). Moreover, this correlation was enhanced in DMRs that were in CpG island shores.

FIG. 5 illustrates that gene expression strongly correlates with reprogramming differentially methylated regions (R-DMRs) at CpG island shores. Red circles represent R-DMRs that are within 2 kb from a CpG island, blue circles represent those that are more than 2 kb away from a CpG island, and black circles represent log ratios for all genes not within (A) 500 bp or (B) 1 kb from the transcriptional start site (TSS) of an annotated gene. The log 2 ratios of fibroblast to iPS expression were plotted against ΔM values (fibroblast minus iPS) for R-DMRs in which one of the two points had approximately no methylation. (A) DMRs that are within 500 bp from a TSS of a gene. (B) DMRs that are within 1 kb from a TSS of a gene.

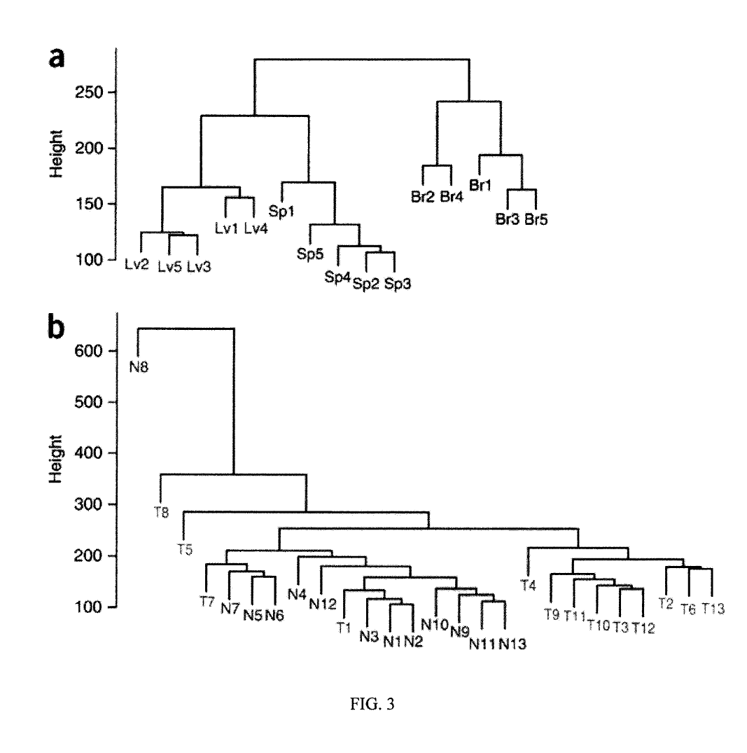

Furthermore, an unsupervised cluster analysis was performed using the R-DMRs to determine to what degree the methylation at these locations distinguished normal brain, liver and spleen from each other. Notably, there was complete separation of these three tissues, indicating that the sites of the methylation changes that occur during reprogramming normally distinguish these disparate tissues (FIG. 3A). In addition, the R-DMRs could largely distinguish normal colonic mucosa from colorectal cancer, indicating that the R-DMRs are also involved in abnormal reprogramming in cancer (FIG. 3B). As a test of significance, none of 1,000 randomly generated lists of the CHARM array regions of equal length and number clustered the tissues as well, as assessed either by whether they yielded a median euclidean distance among samples of a given tissue type at least as low as that found when using the R-DMRs, or yielded a median euclidean distance among samples of different tissue types at least as great as that found when using the R-DMRs. This was true both for the comparison between normal tissues and for the cancer-to-normal-tissue comparison.

FIG. 3 shows that DNA methylation at R-DMRs distinguishes normal tissues from each other and colon cancer from normal colon. (A and B) The M values of all tissues from the 4,401 regions (FDR<0.05) corresponding to R-DMRs (iPS cells compared to parental fibroblasts) were used for unsupervised hierarchical clustering comparing (A) normal brain, spleen and liver (denoted as Br, Sp and Lv, respectively) and (B) colorectal cancer and matched normal colonic mucosa (denoted as T and N, respectively). Notably, all of the normal brain, spleen and liver tissues are completely discriminated by the regions that differ between iPS cells and fibroblasts (R-DMRs). The major branches in the dendrograms correspond perfectly to tissue type. Furthermore, most of the colorectal cancer samples are discriminated from matched normal colonic mucosa by R-DMRs.

The R-DMRs were compared to those obtained in a genome-scale comparison of DNA methylation in colorectal cancer and matched normal colonic mucosa from the same individuals (C-DMRs) as discussed in Irizarry et al. (Nat. Genet. (41)178-186 (2009)). Previously a much smaller number of C-DMRs than T-DMRs (2,707 compared to 16,379) were found, and 45% of the C-DMRs overlapped T-DMRs. Approximately 16% of the R-DMRs in the present study overlapped the C-DMRs of the previous study, whereas only 4.5% on average would be predicted by permutation analysis to overlap (P<0.0001 based on 10,000 permutations) (Table 10). Notably, hypomethylated R-DMRs (iPS compared to fibroblasts) were associated with hypermethylated C-DMRs (cancer compared to normal, P<0.0001 based on 10,000 permutations) (Table 10). Of the 294 DMRs found to overlap between hypomethylated R-DMRs and hypermethylated C-DMRs, 251 (85%) also overlapped bivalent chromatin marks. In contrast, hypermethylated R-DMRs were associated with hypomethylated C-DMRs (P<0.0001 based on 10,000 permutations) (Table 10). Of the 293 DMRs found to overlap between hypermethylated R-DMRs and hypomethylated C-DMRs, only 37 (13%) also overlapped bivalent chromatin marks. Because bivalent chromatin marks are associated with recruitment of Polycomb group proteins, these data suggest that there are two independent epigenetic mechanisms for cell reprogramming and tumorigenesis. One mechanism involves decreased DNA methylation and chromatin modifications at bivalent sites during reprogramming and increased methylation in cancer. The other mechanism involves increased methylation during reprogramming and loss of methylation in cancer.

In summary, it was determined that epigenetic reprogramming of human fibroblasts to iPS cells involves substantial changes in DNA methylation largely affecting the same CpG island shores in T-DMRs that mark normal differentiation. It is notable that the R-DMRs completely distinguish brain from liver from spleen tissues and largely distinguish colon cancer from normal colon tissue. These results provide compelling evidence of the importance of CpG island shores and T-DMRs in both normal development and somatic cell reprogramming. Indeed, the target loci for normal tissue programming, epigenetic reprogramming to pluripotency and aberrant programming of cancers largely overlap. A secondary finding is that certain loci in iPS cells remain incompletely reprogrammed, whereas others are aberrantly reprogrammed, thus establishing that the methylation pattern of iPS cells differs both from those of the parent somatic cells and from those of human ES cells.

These results contrast with prior studies that were primarily directed toward developing powerful new tools to analyze DNA methylation of targeted genomic regions rather than genome-scale studies of iPS cell methylation. The more extensive genome-scale analysis of nine paired sets of iPS cells and parental fibroblasts detected roughly equal levels of hypo- and hypermethylation and revealed the predominant involvement of CpG island shores over islands themselves. The present study reveals a host of loci that represent targets of epigenetic remodeling that are central to somatic cell reprogramming. These R-DMRs include both hypomethylated and hypermethylated regions and are a subset of the previously described T-DMRs and C-DMRs, indicating that these R-DMRs at CpG island shores are critical epigenetic targets for defining cell fate.

Finally, the colocalization of hypomethylated R-DMRs in iPS cells with hypermethylated C-DMRs in cancer and bivalent chromatin marks, and hypermethylated R-DMRs with hypomethylated C-DMRs and the absence of these marks, suggest two parallel mechanisms for epigenetic reprogramming in iPS cells and in cancer, one involving a loss of DNA methylation in iPS and a chromatin-dependent gain of DNA methylation in cancer and the other involving a gain of methylation in iPS and a chromatin-independent loss of DNA methylation in cancer.

### Example II

### Epigenetic Memory in Induced Pluripotent Stem Cells

The following experimental protocols and materials were utilized.

Tissue culture was performed as follows. Bl-iPSC and NP-iPSC were prepare as previously described in Hanna et al. (Cell. (133)250-264. (2008)), Kirov et al. (Genomics (82)433-440 (2003)) and Markoulaki et al. (Nat Biotechnol. (27)169-171. (2009)). The cells were cultured in standard ES maintenance media.

Generation of B-iPSC and F-iPSC was performed as follows. B-iPSC were generated from bone marrow cells collected from one-year-old B6CBAF1 mice. Early progenitor cells (lin−, CD45+, and cKit+) were sorted by FACS (HemNeoFlow Facility at the Dana Farber Cancer Institute) and stained with lineage-specific antibodies (B220; RA3-6B2, CD19; 1D3, CD3; 145-2c11, CD4; GK1.5, CD8; 536.7, Ter119; ter119, Gr-1; RB6-8C5), CD45 specific antibody (30-F11), and cKit antibody (2B8). 10⁵ sorted cells were infected with retrovirus generated from pMXOct4, pMXSox2, pMXK1f4 (as described in Takahashi & Yamanaka, Cell. (126)663-676 (2006)), and pEYK3.1cMyc (as described in Koh et al., Nucleic Acids Res. 30, e142 (2002)) in 6 well dishes with 0.5 ml of each viral supernatant (total 2 ml per well), and spun at 2500 rpm at 20 C for 90 minutes (BenchTop Centrifuge, BeckmanCoulter, Allegra-6R). cMyc was cloned into pEYK3.1 containing two loxp sites to enable removal of the cMyc by Cre treatment. The reprogramming factor-infected cells were plated on to irradiated OP9 feeder cells in 10 cm tissue culture dish in IMDM media (Invitrogen) supplemented with 10% FBS, 1× penicillin/streptomycin/glutamine (Invitrogen), VEGF (R&D Systems, 40 ng/ml), Flt (R&D Systems, 100 ng/ml), TPO (R&D Systems, 100 ng/ml), and SCF (R&D Systems, 40 ng/ml) on day 0. The media were changed on day 2. Cells were collected by media centrifugation and returned to culture during media changes. On day 5, cultured cells were trypsinized and replated on to four 10 cm dishes pre-coated with irradiated mouse embryonic fibroblast in ES maintenance media. Media were changed daily until ES-like colonies were observed. F-iPSC were generated from tail tip fibroblasts of one-year-old B6CBAF1 mice. 10⁶ fibroblast cells were plated onto all wells of a 6 well plate and spin-infected with the four viral supernatants, as for the generation of B-iPSC. Cells were cultured further in DMEM media (Invitrogen) supplemented with 15% FBS, 1× penicillin/streptomycin/glutamine (Invitrogen). On day 5, the cultured cells were trypsinized, and replated in four 10 cm dishes on irradiated mouse embryonic fibroblast with ES maintenance media. Media was changed every day until ES-like colonies were observed.

Differentiation of iPSC to hematopoietic and osteogenic lineages was performed as follows. Hematopoietic colony forming activity of d6 EBs differentiated from pluripotent stem cells was measured in methylcellulose medium with IL3, IL6, Epo, and SCF (M3434, StemCell Tech.) as described in Kyba et al. (Cell (109)29-37 (2002)). Hematopoietic colony type was determined on day 10. Colony identity was confirmed by leukostain analysis of cytospin preparation of the methylcellulose colonies. Osteogenic differentiation was performed by culturing pluripotent stem cells in 15 μl hanging drops (800 cells/drop) in ES differentiation media¹⁶. Embryoid bodies (EB) from hanging drops were collected at 2 days, transferred to a 10 cm dish of non-tissue culture grade plastic with 10⁻⁶ M of retinoic acid, and cultured for 3 days on the shaker (50 rpm) in an incubator. EBs were equally distributed among 3 wells of a 6-well tissue culture dish, and cultured in aMEM media supplemented with 10% FBS, 1× penicillin/streptomycin/glutamine (Invitrogen), 2 nM triiodothyronine, 1× insulin/transferrin/triacostatin A (Gibco, #51300-044). The media were changed every other day. On day 11, one well of each sample was used to measure calcium concentration, osteogenic gene expression (RNA isolation), and for Alizarin Red staining. For Alizarin Red staining, cells were washed with PBS and fixed with 4% paraformaldehyde for 5 minutes at 20 C. Fixed cells were incubated for 15 minutes in Alizarin Red staining solution (Alizarin Red (Sigma, A5533) 2% in H₂O, pH 4-4.3 adjusted with NH₄OH, filtered with 0.45 uM membrane), and washed with Tris-HC1, pH4.0. Elemental calcium concentrations were measured by inductively coupled plasma—atomic emission spectroscopy (ICP-AES, HORIBA Jobin Yvon Activa-M) as described in Nomlru et al. (Anal. Chem. (66)3000-3004 (1994)) at the Center for Materials Science and Engineering at MIT, and three measurements were conducted to obtain mean and standard deviation values. To measure ionized calcium, cells were treated with 5% HNO₃ (for dissolution of calcium molecules) and 10% HClO₄ acid solutions (to remove organic compounds) in a cell culture flask and then briefly sonicated for 10 min. The solution was incubated for >3 hrs on the titer plate shaker. The obtained values were converted to calcium concentration using a reference solution made by Fluka (Calcium Standard for AAS, TraceCERT®), and normalized by 5×10⁵ initiated cells.

Quantitative RT-PCR analysis was performed as follows. The expression levels of osteogenic genes (Runx2, Sp7, and Bglap) were quantified by real-time RT-PCR with Quantifast SYBR Green RT-PCR kit (Qiagen, Hilden, Germany). Total RNAs (2 ug) were reverse-transcribed in a volume of 20 ul by using the SuperScript III First-Strand Synthesis System (Invitrogen, Carlsbad, Calif., USA), and the resulting cDNA was diluted into a total volume of 500 ul. 5 ul of this synthesized cDNA solution was used for analysis. For osteogenic genes, each reaction was performed in a 25 ul volume using the Quantifast SYBR Green RT-PCR kit (Qiagen, Hilden, Germany). The conditions were programmed as follows: initial denaturation at 95 C for 5 min followed by 40 cycles of 10 s at 95 C and 30 s at 60 C, then 1 min at 95 C, 30 s at 55 C, and 30 sec at 95 C. For pluripotent genes, each reaction was performed in a 25 ul volume using the Brilliant SYBR Green QPCR master mix kit (Stratagene, Cedar Creek, Tex., USA). The conditions were programmed as follows: initial denaturation at 95 C for 10 min followed by 40 cycles of 30 s at 95 C, 1 min at 55 C, and 1 min at 72 C, then 1 min at 95 C, 30 s at 55 C, and 30 sec at 95 C. Primers used in the quantitative RT-PCR are listed in Table 12. All of the samples were duplicated, and the PCR reaction was performed using an Mx3005P (Stratagene, Cedar Creek, Tex., USA), which can detect the amount of synthesized signals during each PCR cycle. The relative amounts of the mRNAs were determined using the MxPro program (Stratagene, Cedar Creek, Tex., USA). The amount of PCR product was normalized to a percentage of the expression level of b-Actin. The RT-PCR products of Oct4, Nanog, and b-Actin were also evaluated on 0.8% agarose gels after staining with ethidium bromide. The cycle numbers of the PCR were reduced in order to optimize the difference in band intensities (Oct4, Nanog, and b-Actin were 29, 33, and 28, respectively) (Table 12).

DNA methylation analysis was performed as follows. 5 ug of genomic DNA from each sample was fractionated, digested with McrBC, gel purified, labeled and hybridized to a CHARM microarray as previously described in Irizarry et al. (Nat Genet (41)178-186 (2009)) and Doi et al. (Nat Genet (2009)). For each probe, the averaged methylation values across the same cell type were computed and converted to percent methylation (p). p was used to find regions of differential methylation (Δp) for each pairwise cell type comparison and the absolute area of each region was calculated by multiplying the number of probes by Δp. For data analysis, area value 2 was used as the cutoff to define differentially methylated regions (DMRs). Previous studies indicated that this cutoff corresponds to 5% false discovery rate (Doi et al. unpublished data). Bisulfite pyrosequencing analysis of individual regions was performed as previously described Chan et al., Nat Biotechnol (2009)). Primer sequences are provided in Table 12.

Teratoma and chimera analysis was performed as follows. Teratomas were assessed by injecting 10⁶ undifferentiated cells into the subcutaneous tissue above the rear haunch of Rag2/γC immunodeficient mice (Taconic), and teratoma formation was monitored for 3 months post injection. Collected tumors were processed by the Pathology Core of the Dana-Farber/Harvard Cancer Center. Chimera analysis of pluripotent cells was conducted by injecting GFP+ or GFP− cells into blastocysts isolated from C57BL/6 (GFP− or GFP+) embryos, which were collected at the two-cell stage. The fertilized embryo was collected from the oviduct and cultured in KSOM media (Specialty Media). A mouse strain expressing GFP from the human ubiquitin promoter (Jackson Laboratory) was used to ensure maximum expression in various tissues, and enabled injected cells to be distinguished from host cells. The reconstituted blastocysts were implanted into 2.5 day pseudopregnant CD1 females. Chimeras were allowed to develop to adulthood to gauge skin chimerism and germ cell transmission, or were dissected at embryonic day 12.5 to isolate gonad, liver, heart, and MEF for flow analysis. Gonads were stained with SSEA1 antibody (Hybridoma Bank) for 1 hour, and treated with APC-conjugated mouse IgM antibody (BD Pharmingen, #550676) to detect SSEA1 positive germ cells by flow cytometry (LSRII, BD Biosciences, Hematology/Oncology Flow Cytometry Core Facility of Children's Hospital Boston).

Generation of NSC-NP-iPSC, B-NP-iPSC, and NP-iPSC-TSA-AZA was performed as follows. Neural Progenitor (NP) iPSC harboring integrated proviruses carrying the four reprogramming factors described in Turker (Oncogene (21)5388-5393 (2002)) were differentiated to neural stem cells (NSC) as described in Conti et al. (PLoS Biol. 3, e283. (2005)). Reprogramming factors in cultured NSC were induced by doxycycline, and colonies expressing GFP from the nanog-reporter were selected to yield NSC-NP-iPSC. NP-iPSC from blood lineages (B-NP-iPSC) were obtained by differentiating the NP-iPSC via EB for 6 days, infecting with HoxB4ERT retrovirus as in Schiedlmeier et al. (Proc Natl Acad Sci U S A. (104)16952-16957 (2007)), and co-culturing on OP9 in the presence of 4-hydroxytamoxifen (4-HT) to enable isolation of hematopoietic cells as described in Kyba et al. (Cell 109, 29-37 (2002)). Day 15 hematopoietic cells were harvested, stained with CD45+ (BD Pharmingen, #557659), and sorted for hematopoietic cells. Only minimal hematopoietic colonies were observed on OP9 culture in the absence of 4-HT. Harvested CD45+ hematopoietic cells were induced by doxycycline and colonies expressing GFP from the nanog-reporter were selected to yield B-NP-iPSC. Methylcellulose hematopoietic colony analysis was conducted in the absence of 4-HT as a negative control. The dissociated EBs (2×10⁵ cells) from NP-iPSC were infected with HoxB4-ERT virus and then plated on methylcellulose media. Only 1.7+/−1.2 colonies (n=3) were formed in the absence of hydroxytamoxifen, which indicates the limited functional HoxB4 expression in the absence of 4-HT. NP-iPSC-TSA-AZA cells were isolated by treating cells for 9 days with Trichostatin A (TSA, 100 nM) and 5-azacytidine (AZA, 1 mM), in 3-day cycles: drug treatment occurred on two consecutive days, followed by one day of non-treatment. Undifferentiated colonies were recovered to conduct methylcellulose analysis. Wnt3a (R&D System, 1324-WN-002/CF, 40 ng/ml) was added to EB culture media between day 2 and 4, and hematopoietic potential was tested by plating on methylcellulose media as described above.

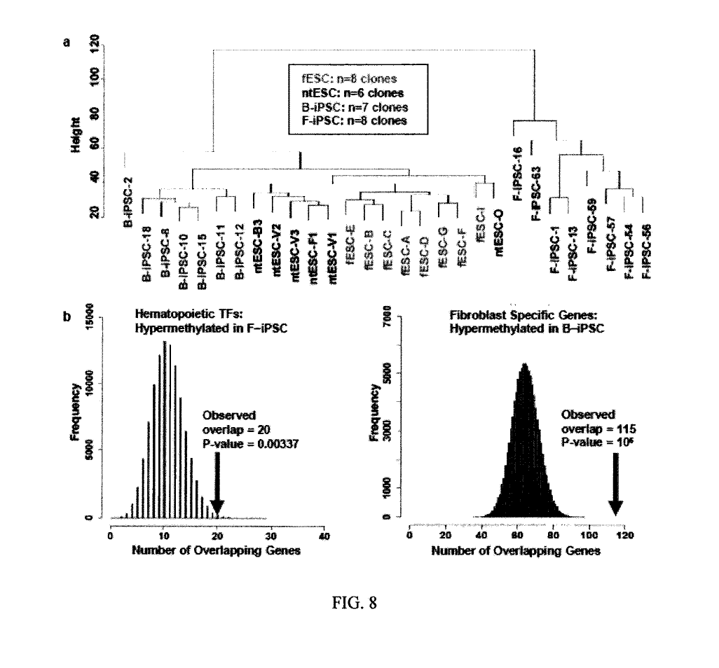

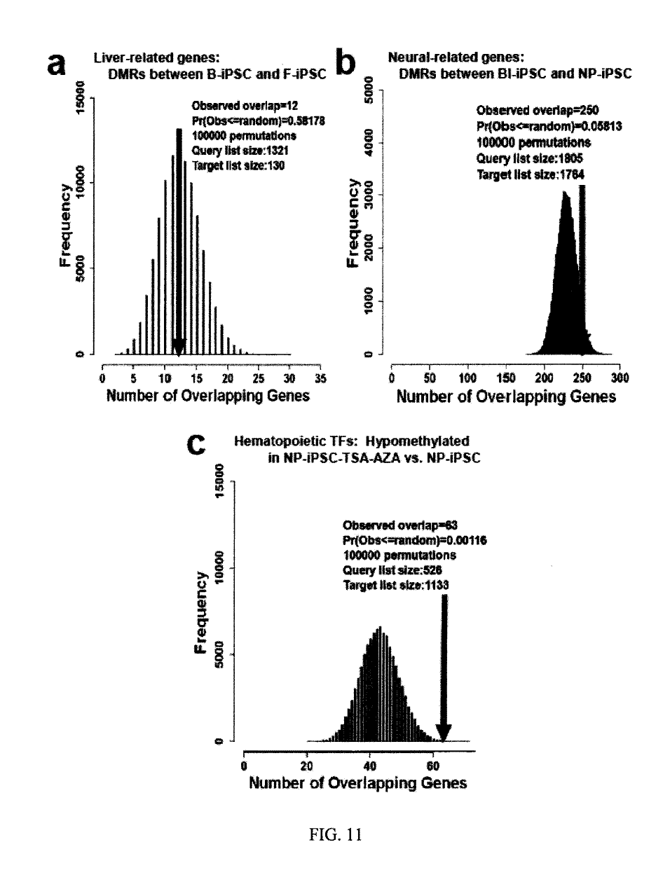

Gene enrichment analysis was performed as follows. A permutation approach was taken to assess the enrichment of hematopoiesis and fibroblast related genes in DMRs. Gene lists were derived from MSigDB (broadinstitute.org/gsea/msigdb/index.jsp) for FIG. 11C, and cell-type signatures are described in Cahan et al. (manuscript in preparation (2010)) for all other enrichment analyses. To identify cell-type signatures, gene expression profiles of more than 80 distinct cell types were downloaded from Gene Expression Omnibus, normalized, and searched for sets of genes that exhibit cell type-specific expression patterns, using the template matching method described in Pavlidis et al. (Genome biology 2, RESEARCH0042 (2001)). Enrichment P-values were calculated as the number of times that a random selection of genes from the 13,931 profiled met or exceeded the observed overlap based on 100,000 random selections. The number of randomly selected genes was the same as the number of genes in the DMR list. FIG. 8B (left panel): 20/74 hematopoiesis-related transcription factors are among the 1,997 genes hypermethylated in F-iPSC vs B-iPSC (P-value=0.00337). FIG. 8B (right panel): 115/562 fibroblast-specific genes are among the 1,589 genes hypermethylated in B-iPSC vs F-iPSC (P-value=0.00001). FIG. 11A: 12/130 liver-specific genes are among the 1,321 differentially methylated in F-iPSC vs B-iPSC (P-value=0.58178). FIG. 11B: 250/1764 neural-specific genes are among the 1,805 differentially methylated in Bl-iPSC vs. NP-iPSC (P-value=0.05813). FIG. 11C: 63/526 genes up-regulated in hematopoietic stem cells are among the 1,133 genes hypomethylated in NP-iPSC-TSA-AZA vs NP-iPSC (P-value=0.00116).

Transcription factor reprogramming differs markedly from nuclear transfer, particularly with regard to DNA demethylation, which commences immediately upon transfer of a somatic nucleus into ooplasm, but occurs over days to weeks during the derivation of iPSC. Because demethylation is a slow and inefficient process in factor-based reprogramming, it was postulated that residual methylation might leave iPSC with an “epigenetic memory,” and that methylation might be more effectively erased by nuclear transfer. A comparison of the differentiation potential and genomic methylation of pluripotent stem cells (iPSC, ntESC, and fESC) was performed and evidence that iPSC indeed retain a methylation signature of their tissue of origin was found.

Initially, it was sought to compare the in vivo engraftment potential of hematopoietic stem cells derived from fESC, ntESC, and iPSC in a mouse model of thalassemia. However, even in vitro different blood-forming potential was strikingly observed; thus, focus was placed here instead on understanding this phenomenon. The initial set of pluripotent stem cells were derived from the hybrid C57BL/6×CBA (B6/CBAF1) strain carrying a deletion in the beta-globin locus as described in Skow et al. (Cell (34)1043-1052. (1983)), which is otherwise irrelevant to this study (FIG. 1a). fESC cells were isolated from naturally fertilized embryos and derived ntESC cells from nuclei of dermal fibroblasts as described in Blelloch et al. (Stem Cells. (24)2007-2013 (2006)). Early bone marrow cells were infected (Kit+, Lin−, CD45+) or dermal fibroblasts from aged mice with retroviral vectors carrying Oct4, Sox2, K1f4, and Myc, and selected blood-derived and fibroblast-derived iPSC colonies (B-iPSC, F-iPSC). Hematopoietic progenitors and fibroblasts yielded a comparable frequency of reprogrammed colonies (0.02%), which consistent with prior reports (Li et al., Nature (460)1136-1139 (2009)), was lower than the yield from fibroblasts of a juvenile mouse (0.1%). The fESC, ntESC, and iPSC lines were characterized for expression of Oct4 and Nanog by immunohistochemistry, and demonstrated multi-lineage differentiation potential in teratomas (data not shown). By criteria typically applied to human samples and appropriate for a therapeutic model as discussed in Daley et al. (Cell Stem Cell (4)200-201; author reply 202 (2009)), all stem cell lines manifest pluripotency.

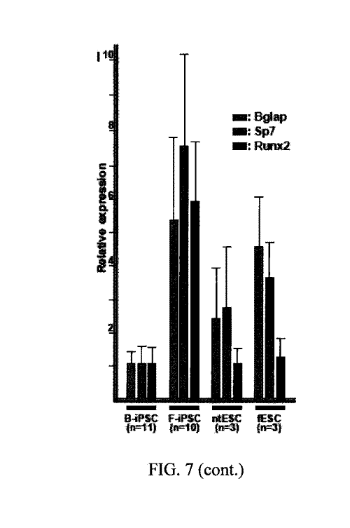

Differentiation of pluripotent stem cells. To test blood potential, multiple pluripotent stem cell clones were differentiated into embryoid bodies (EBs), dissociated cells, and assayed for hematopoietic colony forming cells as described in Kyba et al. (Cell (109)29-37 (2002)). All pluripotent cells generated comparable EBs but markedly different numbers of hematopoietic colonies. Consistently, blood-derived B-iPSC yielded more hematopoietic colonies than F-iPSC (FIG. 7A). Hematopoietic colony formation from ntESC and fESC were higher than the iPSC lines.

Differentiation into osteoblasts was then tested, a mesenchymal lineage that can be derived from fibroblasts as described in Bourne et al. (Tissue Eng. (10)796-806 (2004)) and Wdziekonski et al. (Curr Protoc Cell Biol. Chapter 23, Unit 23.24. (2007)). By alizarin red staining, a marker of osteogenic cells, F-iPSC produced more sharply defined osteogenic colonies (data not shown), deposited more elemental calcium (FIG. 7B), and showed higher expression of three osteoblast-associated genes (FIG. 7C) than B-iPSC. By these criteria, F-iPSC show enhanced osteogenic potential, reflecting a propensity to differentiate towards a mesenchymal lineage. In contrast, ntESC cells behaved comparably to fESC in hematopoietic and osteogenic assays.

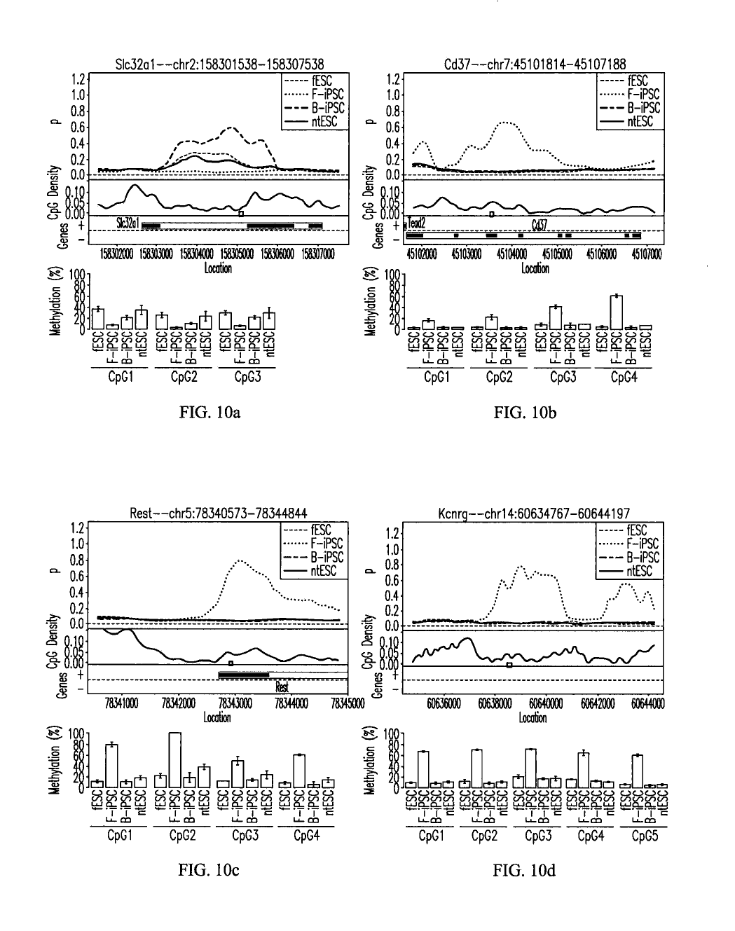

DNA methylation of pluripotent stem cells. It was hypothesized that the different pluripotent cells might harbor different patterns of genomic DNA methylation; thus, Comprehensive High-throughput Array-based Relative Methylation (CHARM) analysis was performed, which interrogates ˜4.6 million CpG sites, including almost all CpG islands and nearby sequences termed shores as discussed in Irizarry et al. (Genome Res (18)780-790 (2008)) and Irizarry et al. (Nat Genet (41)178-186 (2009)), but does not assess non-CpG methylation. It was determined that the number of differentially methylated regions (DMRs) between pair-wise comparisons, using a threshold area cutoff of 2, corresponding to a 5% false discovery rate (FDR²²; Table 13A). By this analysis, ntESC were most similar to fESC (only 229 DMRs), whereas F-iPSC differed most extensively (5304 DMRs). Relative to fESC, hypermethylated DMRs predominated for F-iPSC (3349=63%) and B-iPSC (516=74%). Highlighting their functional differences, 5202 DMRs were identified between B-iPSC and F-iPSC. The results of CHARM analysis were confirmed by bisulfite pyrosequencing of multiple loci (FIG. 10).

Unsupervised hierarchical clustering of DMRs between B-iPSC and F-iPSC easily distinguished iPSC from ntESC and fESC, which cluster together (FIG. 8A). B-iPSC cluster nearer to ntESC and fESC than do F-iPSC, which represent a strikingly separate cluster. These data indicate that the methylation patterns of ntESC are more like fESC than are either iPSC.

Several lines of evidence support a mechanistic link between differential methylation and hematopoietic propensity of iPSC lines. First, literature survey of genes for the top 24 DMRs that distinguish B-iPSC and F-iPSC links 11 to hematopoiesis and 3 to osteogenesis (Table 14). Of the 11 hematopoietic loci, 10 are hypermethylated in F-iPSC relative to B-iPSC. Second, of 74 hematopoietic transcription factors as described in Cahan et al. (manuscript in preparation (2010)), 20 are in or near DMRs that are hypermethylated in F-iPSC versus B-iPSC, twice that predicted by chance (p=0.0034; FIG. 8B left panel, FIG. 11A, and Table 15). Similarly, of 764 fibroblast-specific genes, 115 are hypermethylated in B-iPSC, twice that predicted by chance (p=10⁻⁵; FIG. 8B right panel). Given the correlation between methylation and transcriptional silencing, the data suggested that iPSC harbor epigenetic marks antagonistic to cell lineages distinct from the donor cell type.

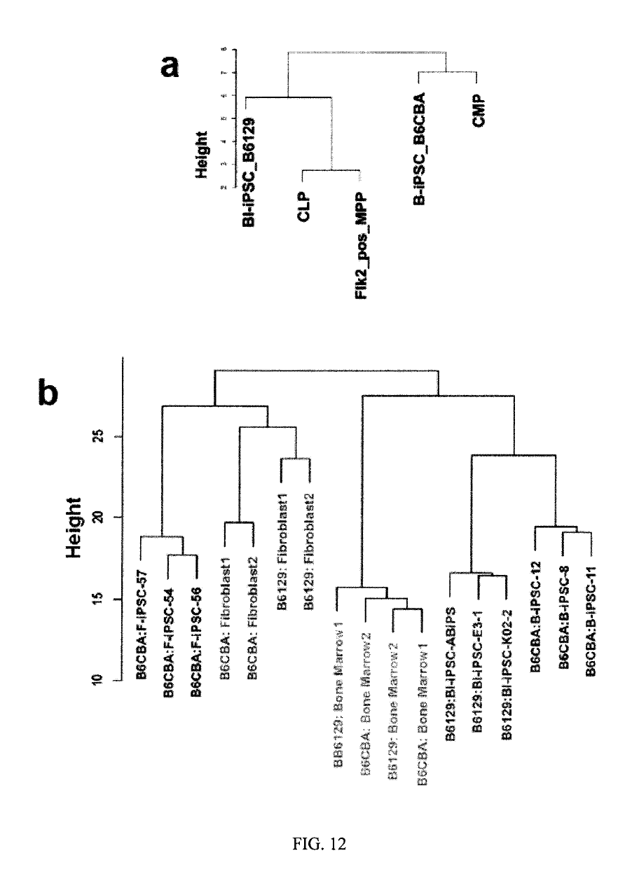

It was asked whether DMRs that distinguish B-iPSC from fESC might allow one to identify their hematopoietic lineage of origin. In a separate CHARM experiment, genome-wide methylation in highly purified multipotent and lineage-specific hematopoietic progenitors was examined. Comparing DMRs in B-iPSC to those that define hematopoietic progenitors, it was observed that B-iPSC cluster alongside Common Myeloid Progenitors (CMP) and distant from Common Lymphoid Progenitors (CLP; FIG. 12a and Table 16), which is notable given that B-iPSC were derived from Kit+, lineage-negative myeloid marrow precursors. Next, it was asked whether the tissue of origin (bone marrow vs fibroblast) could be identified by the methylation state of tissue specific DMRs in F-iPSC, B-iPSC, and Bl-iPSC (a B lymphocyte-derived iPSC line described below). Using DMRs that distinguish fibroblast and bone marrow, and examining methylation in iPSCs and somatic cells from two different genetic backgrounds (B6CBA and B6129), it was determened that F-iPSC cluster alongside fibroblasts, and distant from bone marrow (FIG. 12B). Similarly, the hematopoietic-derived B-iPSC and Bl-iPSC grouped with somatic cells from bone marrow. Thus, residual methylation indicates the tissue of origin of iPSC, and for blood-derivatives even their precise lineage, further supporting the phenomenon of epigenetic memory in iPSC.

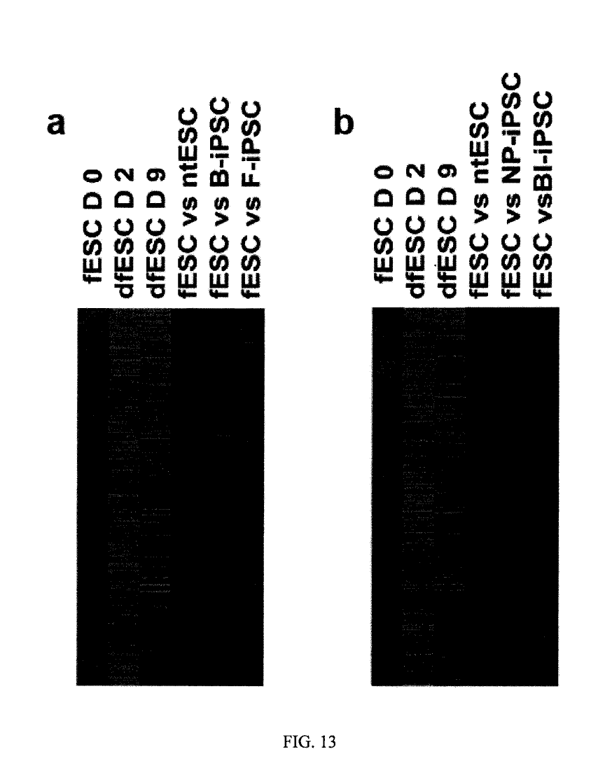

Reprogrammed state of iPSC and ntESC. It was postulated that the differing methylation signatures of B-iPSC, F-iPSC, and ntESC reflect disparate reprogramming, and confirmed this by two independent computational analyses. First, DMRs that distinguish B-iPSC, F-iPSC, and ntESC from fESC were overlapped with genes specifically expressed in undifferentiated murine fESC described in Perez-Iratxeta et al. (FEBS Lett (579)1795-1801 (2005)). By this analysis, ntESC showed the fewest DMRs at loci corresponding to the most highly expressed fESC-specific genes, and B-iPSC showed fewer DMRs at these loci than F-iPSC (FIG. 13A). Second, DMRs were overlapped with the DNA binding locations for seven transcription factors that compose a core protein network of pluripotency described in Kim et al. (Cell. (132)1049-1061 (2008)), and found the fewest DMRs at core transcription factor binding sites in ntESC, and less overlap in B-iPSC than in F-iPSC (Table 17). These analyses indicate that F-iPSC harbor more residual methylation than B-iPSC at loci directly linked to the gene expression and pluripotency networks of fESC, whereas ntESC show the least differential methylation and appear closest to fESC at these critical loci.

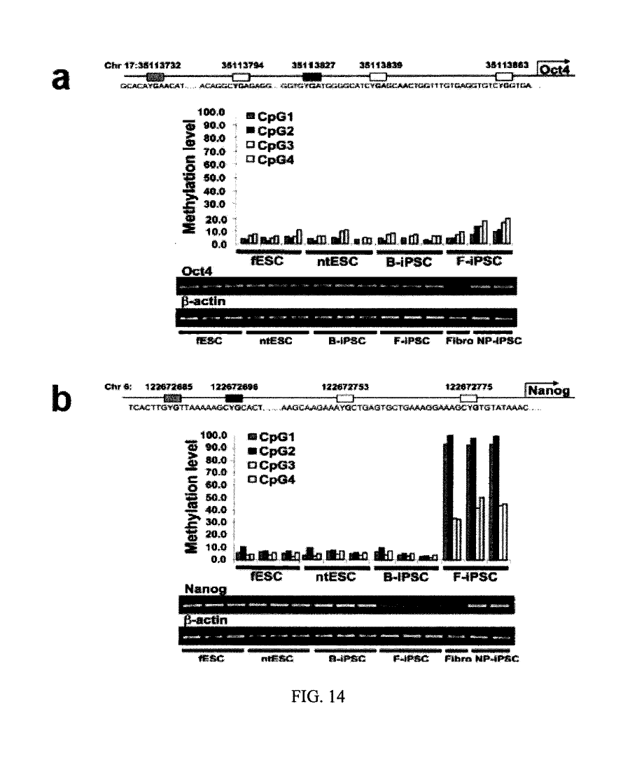

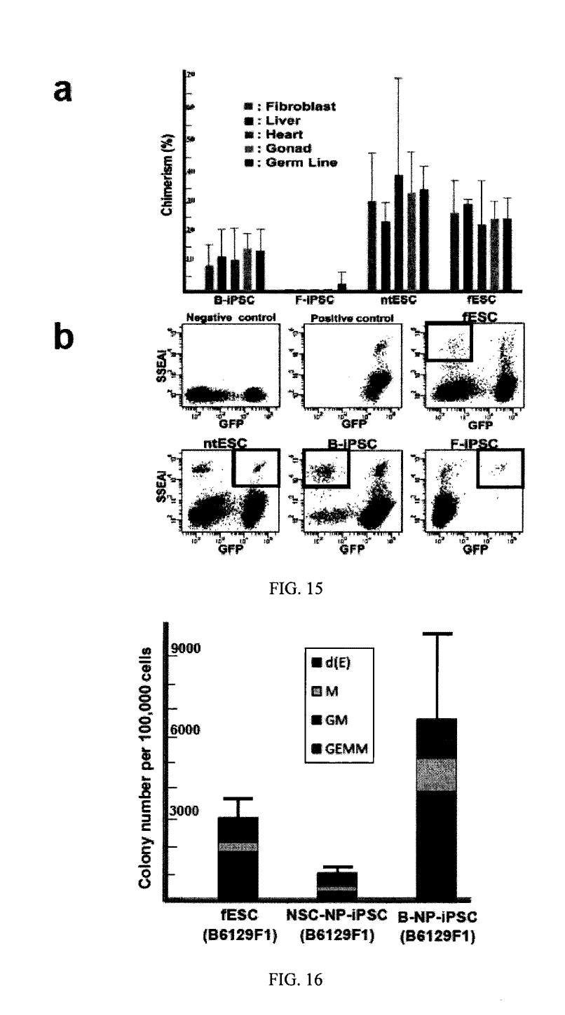

Further analysis of Oct4 and Nanog indicates that although both are detected by immunohistochemistry in B-iPSC and F-iPSC (data not included), Oct4 mRNA is fully expressed from a demethylated promoter in both types of iPSC, whereas Nanog mRNA is sub-optimally expressed from a promoter that retains considerable methylation in F-iPSC (FIG. 14). When assessed by blastocyst chimerism, B-iPSC contribute to all tissues, including the germ line, whereas F-iPSC contribute only poorly (FIG. 15A), although they can be found in SSEA1+ germ cells of the gonadal ridge (FIG. 15B). Thus, while both B-iPSC and F-iPSC generate robust multi-lineage teratomas, satisfying criteria for pluripotency typically applied to human cells, broader functional assessments available in the mouse system confirm their differential degree of reprogramming. In this comparison of iPSC derived from accessible tissues of aged adult mice, bone marrow yields stem cells with superior features of pluripotency, but neither iPSC is equivalent to ntESC or fESC.

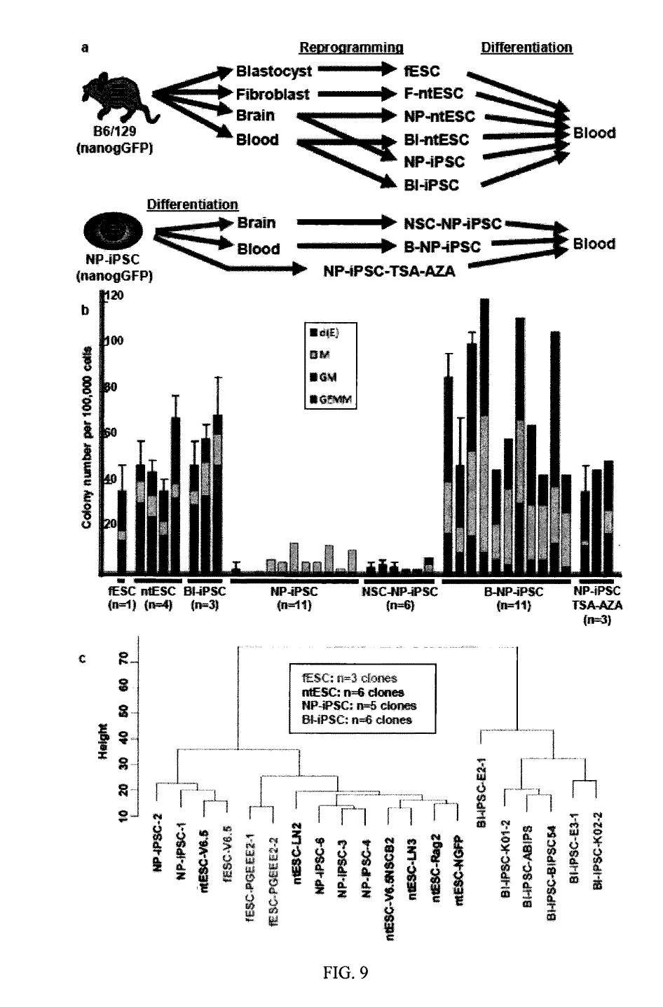

Stringently-defined pluripotent stem cells. To determine if blood-forming potential differs among cell lines that satisfy more stringent criteria for pluripotency, lines derived from a uniform genetic background (B6/129F1) that all express a Nanog-eGFP reporter gene, and for which pluripotency was demonstrated by blastocyst chimerism and transmission through the germ line were analyzed (FIG. 9A, upper schema; Table 18). These studies involve “secondary” iPSC lines derived from neural progenitor cells (NP-iPSC) and B-lymphocytes (B1-iPSC) of mice chimerized with iPSC carrying proviruses that express doxycycline-inducible reprogramming factors from identical proviral integration sites. NP-iPSC and Bl-iPSC were compared to ntESC generated from neural progenitor cells (NP-ntESC), blood progenitor cells (B-ntESC), and fibroblasts (F-ntESC), as well as fESC.

All cell lines were differentiated into embryoid bodies and assayed for hematopoietic colony forming activity as described in Kyba et al. (Cell (109)29-37 (2002)). Across multiple clones, higher blood forming potential was observed of iPSC derived from B lymphocytes (B1-iPSC) than from neural progenitors (NP-iPSC; FIG. 9B). In contrast, it was observed that ntESC, regardless of tissue origin (fibroblasts, neural progenitors, or T-cells), and fESC displayed an equivalently robust blood forming potential (FIG. 9B). In this independent set of iPSC lines, qualified as pluripotent by stringent criteria, consistent differences in blood formation were again observed, with blood derivatives showing more robust hematopoiesis in vitro than neural derivatives.

Resetting differentiation propensity. Finally, it was asked whether the poor blood-forming potential of NP-iPSC by differentiation into hematopoietic lineages could be rescued, followed by a tertiary round of reprogramming back to pluripotency by doxycycline induction of the endogenous reprogramming factors (FIG. 9A, lower schema). As a control, NP-iPSCs were differentiated into neural stem cells, followed by tertiary reprogramming to pluripotency. Resulting iPSC clones were selected for expression of the Nanog-eGFP reporter and shown to express Oct4 and Nanog by immunohistochemistry (data not included) and to chimerize murine blastocysts (data not included). The tertiary blood-derived B-NP-iPSC showed higher hematopoietic colony-forming potential than the tertiary NSC-NP-iPSC (FIG. 9B), and generated larger hematopoietic colonies with more cells per colony (FIG. 16B). These data indicate that the poor blood-forming potential of secondary NP-iPSC can be enhanced by differentiation into hematopoietic progeny, followed by tertiary reprogramming. In contrast, tertiary reprogramming via neural intermediates yields iPSC that retain poor hematopoietic potential.

The reduced blood potential of NP-iPSC might be explained by residual epigenetic marks that restrict blood fates or a lack of epigenetic marks that enable blood formation. Determination of whether treatment of NP-iPSC with pharmacologic modulators of gene expression and DNA methylation might reactivate latent hematopoietic potential was sought. NP-iPSC were treated in vitro with Trichostatin A (TSA), a potent inhibitor of histone deacetylase, and 5-azacytidine (AZA), a methylation-resistant cytosine analogue. After 18 days of drug treatment, the resulting cells displayed higher blood forming activity (NP-iPSC-TSA-AZA; FIG. 9B). For unclear reasons, tertiary reprogramming through blood intermediates or drug treatment of NP-iPSC produced altered ratios of colony sub-types, perhaps suggesting different efficiencies of lineage reprogramming.

Methylation in secondary and tertiary iPSC. CHARM was used to examine the methylome of the germ-line competent pluripotent stem cells, the tertiary reprogrammed B-NP-iPSC and NSC-NP-iPSC, and the drug-treated NP-iPSC (FIG. 9A). In pair-wise comparisons (Table 13B), the NP-iPSC showed only a small number of DMRs relative to fESC (553), fewer than the numbers of DMRs distinguishing ntESC from fESC (679), indicating that selection using the Nanog-GFP reporter and derivation from young donor tissue yields more equivalently reprogrammed cells. Despite equivalent Nanog-GFP expression, B lymphocyte-derived Bl-iPSC harbored more DMRs (1485) relative to fESC than did the NP-iPSC. Cluster dendrogram analysis, employing the most variable DMRs that distinguish Bl-iPSC and NP-iPSC, showed NP-iPSC to be more similar to fESC than are Bl-iPSC, which represent a distinct cluster (FIG. 9C). These data suggest that neural progenitors are more completely reprogrammed to an ESC-like state than blood donor cells. Cluster dendrogram analysis failed to distinguish among NP-iPSC, ntESC, and fESC, but assessment of the overlap of DMRs with loci for highly expressed ESC-specific genes and core pluripotency transcription factor binding sites indicated differences among these three pluripotent cell types, and reveal that ntESC have the fewest DMRs affecting these critical loci (FIG. 13B).

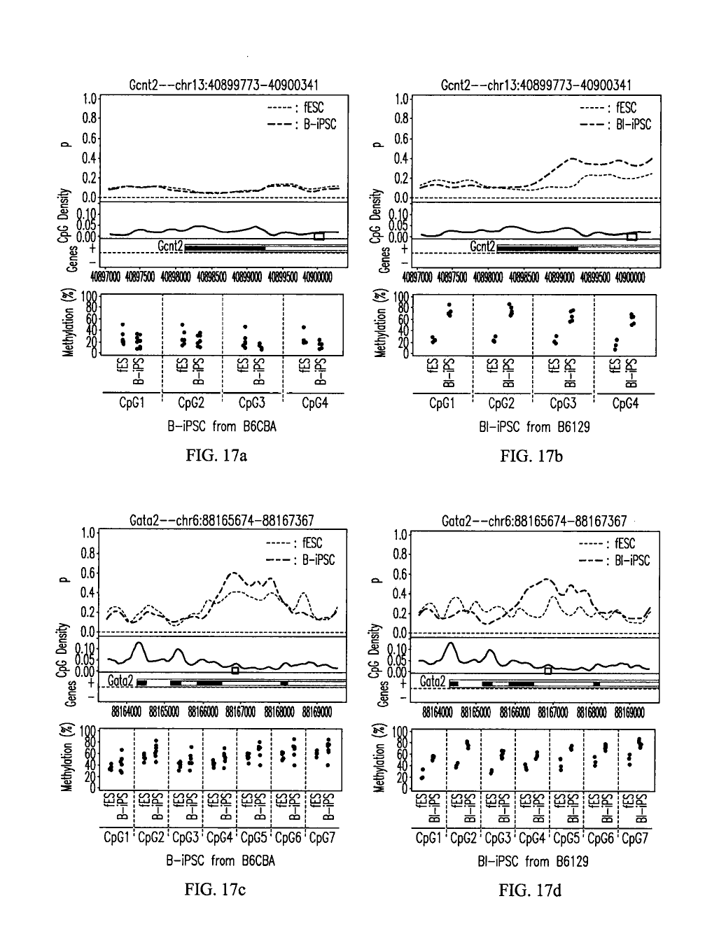

Relative to fESC, hypermethylated DMRs predominated in NP-iPSC and Bl-iPSC (417 (75%) and 1423 (96%), respectively; Table 13B), confirming that even when pluripotency is documented by stringent criteria, iPSC retain residual methylation. By analysis of overlapping DMRs, Bl-iPSC cluster with progenitors of the lymphoid lineage (CLP) rather than the myeloid lineage (FIG. 9A; Table 16). To illustrate this point, the Gcnt2 gene, which encodes the enzyme responsible for the blood group I antigen, and Gata2, a regulator of hematopoiesis and erythropoiesis, are both hypermethylated and transcriptionally silent in the lymphoid lineage. Bl-iPSC showed hypermethylation at these loci relative to fESC, whereas the myeloid-derived B-iPSC did not (FIG. 17). Thus, a methylation signature correctly identifies the blood lineage of origin of B-lymphocyte derived iPSC. Furthermore, it was found that neural-related genes tended to be differentially methylated between Bl-iPSC and NP-iPSC (FIG. 8B). Treatment of NP-iPSC with TSA and AZA enhances blood-forming potential and increases hypomethylated DMRs (626; Table 13C). Significant overlap was found between these DMRs and genes enriched in mouse hematopoietic stem cells (MSigDG signature STEMCELL_HEMATOPOIETIC_UP; FIG. 11C), suggesting that drug treatment erases inhibitory methylation signatures at hematopoietic loci.

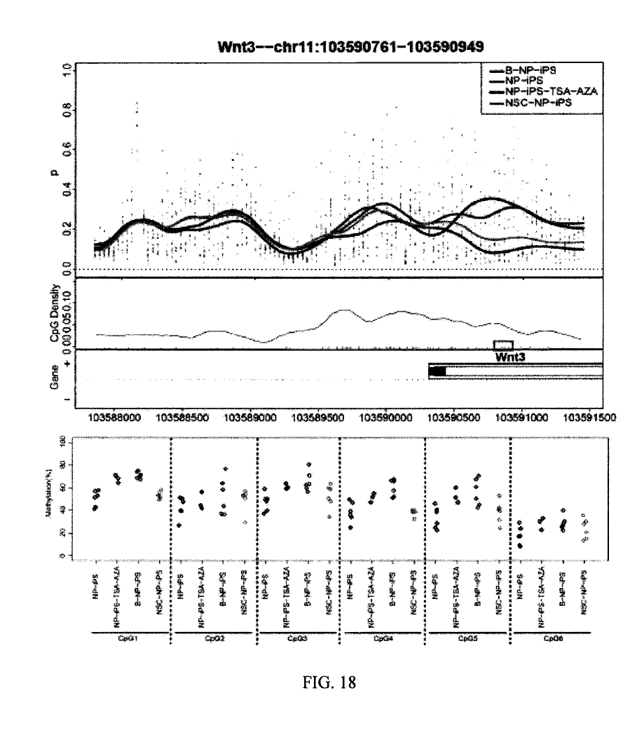

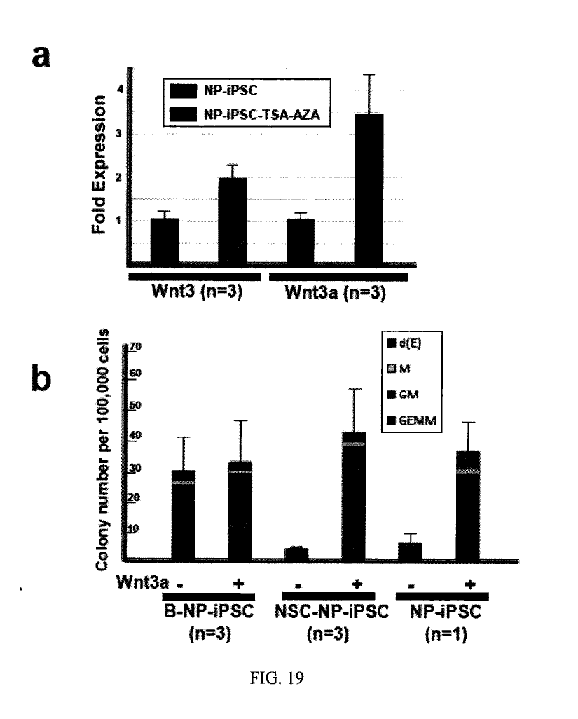

DMRs in iPSCs with high hematopotietic potential (B-NP-iPSC and NP-iPSC-TSA-AZA) were compared to those with low hematopoietic potential (NP-iPSC and NSC-NP-iPSC), and it was found that B-NP-iPSC and NP-iPSC-TSA-AZA harbored higher gene-body methylation of Wnt3 (FIG. 18), a gene which along with its homologue Wnt3a plays a major role in blood development from fESC. The blood-deficient NP-iPSC and NSC-NP-iPSC lines lacked gene body methylation. While promoter methylation is repressive, gene body methylation is seen in active genes. When iPSC were differentiated into embryoid bodies, the blood-prone NP-iPSC-TSA-AZA showed higher levels of Wnt3/3a expression than the blood-deficient NP-iPSC (FIG. 19A). Interestingly, supplementation of the culture media with Wnt3a during embryoid body differentiation restored blood-forming potential in the blood-deficient NP-iPSC and NSC-NP-iPSC lines, but had little effect on the already robust hematopoietic potential of B-NP-iPSC (FIG. 19B). Albeit preliminary, these data correlate differential gene body methylation and expression of the Wnt3 locus with enhanced blood-forming potential in iPSC lines.

Discussion

It is demonstrated herein that iPSC retain an epigenetic memory of their tissue of origin. The data reveal several important principles that relate to the technical limitations inherent in the process of reprogramming, and which in practice influence the differentiation propensity of specific isolates of iPSC.

First, tissue source influences the efficiency and fidelity of reprogramming. From aged mice, blood cells were reprogrammed more closely to fESC than dermal fibroblasts, which yielded only incompletely reprogrammed cells. Neural progenitor-derived iPSC were most similar to fESC, consistent with previous evidence that such cells can be reprogrammed with fewer transcription factors. Whereas neural progenitors are not readily accessible, iPSC can be generated by direct reprogramming of human blood.

Second, analysis of DNA methylation reveals substantial differences between iPSC and embryo-derived ESC (ntESC and fESC). iPSC derived from non-hematopoietic cells (neural progenitors and fibroblasts) retain residual methylation at loci required for hematopoietic fate, which manifests as reduced blood-forming potential in vitro. Residual methylation signatures link iPSC to their tissue of origin, and even discriminate between the myeloid and lymphoid origins of blood-derived iPSC. Prior studies reporting residual hypermethylation in iPSC did not establish a link between DMRs at specific loci, tissue of origin, and altered differentiation potential. While residual methylation is mostly repressive, it was shown for Wnt3 that residual gene body methylation in blood-derived iPSC is associated with enhanced blood potential. Interestingly, the poor blood potential of neural progenitor-derived iPSC, which lack this epigenetic mark and express lower levels of endogenous Wnt3, can be enhanced by supplementing differentiating cultures with exogenous Wnt3a cytokine, indicating that manipulating culture conditions can overcome epigenetic barriers.

Third, the differentiation propensity and methylation profile of iPSC can be reset. When blood-deficient neural progenitor-derived iPSC (NP-iPSC) are differentiated into blood and then reprogrammed to pluripotency, their blood-forming potential is markedly increased. Alternatively, treatment of NP-iPSC with chromatin-modifying compounds increases blood-forming potential and is associated with reduced methylation at hematopoietic loci. For some applications, epigenetic memory of the donor cell may be advantageous, as directed differentiation to specific tissue fates remains a challenge.

Fourth, nuclear transfer-derived ESC are more faithfully reprogrammed than most iPSC generated from adult somatic tissues. Like the immediate and rapid demethylation of the sperm pronucleus following fertilization, somatic nuclei are rapidly demethylated by nuclear transfer into ooplasm, prompting speculation that the egg harbors an active demethylase. In contrast, demethylation is a late phenomenon in factor-based reprogramming, and likely occurs passively. Studying how ooplasm erases methylation might identify biochemical functions that would enhance factor-based reprogramming Failure to demethylate pluripotency genes is associated with intermediate or partial states of reprogramming, and knock-down of the maintenance methyltranferase DNMT1 or treatment with the demethylating agent 5-AZA can convert intermediate states to full pluripotency. Demethylation appears passage dependent, and reprogramming efficiency correlates with the rate of cell division and the passage number. In these experiments, pluripotent stem cells of comparable low passage number were compared (Table 17), but continued serial passage may homogenize the differentiation potential of pluripotent cell types.

The mRNA expression program of iPSC and fESC are strikingly similar. Minor differences in mRNA and microRNA expression have been reported, but removal of transgenes reduces the differences. The Dlk1-Dio3 locus, whose expression correlates with capacity to generate “all-iPSC” mice, is not differentially methylated and expressed in at least some iPSC lines that manifest epigenetic memory (our unpublished observations). Thus even the most stringently-defined iPSC might retain epigenetic memory. Importantly, differences between iPSC and fESC may not manifest until differentiation, when the specific loci that retain residual epigenetic marks are expressed, influencing cell fates. Methylation is but one molecular feature of “epigenetic memory” in iPSC. Faulty restoration of bivalent domains, which mark developmental loci with both active and repressive histone modifications, and loss of pioneer factors, which in fESC and iPSC occupy enhancers of genes expressed only in differentiated cells, represent two other potential mechanisms.

Although ideal, generic iPSC may be functionally and molecularly indistinguishable from fESC, it is shown in practice that even rigorously selected iPSC can retain epigenetic marks characteristic of the donor cell that influence differentiation propensity. Epigenetic differences are unlikely to be essential features of iPSC, but rather reflect stochastic variations associated with the technical challenges of achieving complete reprogramming. Given that reporter genes for selecting human iPSC are lacking, and one cannot qualify their pluripotency by assaying embryo chimerism, the behavior of human cells will likely be influenced by epigenetic memory. Human ESC can also manifest variable differentiation potential.

Tables

Lengthy table referenced here
US20120164110A1-20120628-T00001
Please refer to the end of the specification for access instructions.

Lengthy table referenced here
US20120164110A1-20120628-T00002
Please refer to the end of the specification for access instructions.

Lengthy table referenced here
US20120164110A1-20120628-T00003
Please refer to the end of the specification for access instructions.

Lengthy table referenced here
US20120164110A1-20120628-T00004
Please refer to the end of the specification for access instructions.

Lengthy table referenced here
US20120164110A1-20120628-T00005
Please refer to the end of the specification for access instructions.

Lengthy table referenced here
US20120164110A1-20120628-T00006
Please refer to the end of the specification for access instructions.

Lengthy table referenced here
US20120164110A1-20120628-T00007
Please refer to the end of the specification for access instructions.

Lengthy table referenced here
US20120164110A1-20120628-T00008
Please refer to the end of the specification for access instructions.

Lengthy table referenced here
US20120164110A1-20120628-T00009
Please refer to the end of the specification for access instructions.

Lengthy table referenced here
US20120164110A1-20120628-T00010
Please refer to the end of the specification for access instructions.

Lengthy table referenced here
US20120164110A1-20120628-T00011
Please refer to the end of the specification for access instructions.

Lengthy table referenced here
US20120164110A1-20120628-T00012
Please refer to the end of the specification for access instructions.

Lengthy table referenced here
US20120164110A1-20120628-T00013
Please refer to the end of the specification for access instructions.

Lengthy table referenced here
US20120164110A1-20120628-T00014
Please refer to the end of the specification for access instructions.

Lengthy table referenced here
US20120164110A1-20120628-T00015
Please refer to the end of the specification for access instructions.

Lengthy table referenced here
US20120164110A1-20120628-T00016
Please refer to the end of the specification for access instructions.

Lengthy table referenced here
US20120164110A1-20120628-T00017
Please refer to the end of the specification for access instructions.

Lengthy table referenced here
US20120164110A1-20120628-T00018
Please refer to the end of the specification for access instructions.

Although the invention has been described with reference to the above example, it will be understood that modifications and variations are encompassed within the spirit and scope of the invention. Accordingly, the invention is limited only by the following claims.

LENGTHY TABLES
The patent application contains a lengthy table section. A copy of the table is available in electronic form from the USPTO web site (http://seqdata.uspto.gov/?pageRequest=docDetail&DocID=US20120164110A1). An electronic copy of the table will also be available from the USPTO upon request and payment of the fee set forth in 37 CFR 1.19(b)(3).

What is claimed is:

1. A method of identifying an induced pluripotent stem (iPS) cell comprising:
comparing the methylation status of one or more nucleic acid sequences of a putative iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of an iPS cell, wherein a similarity in methylation status is indicative of the putative cell being an iPS cell.

2. The method of claim 1, wherein the one or more nucleic acid sequences are within a gene.

3. The method of claim 1, wherein the one or more nucleic acid sequences are upstream or downstream of a gene.

4. The method of claim 1, wherein the one or more nucleic acid sequences are selected from the group consisting of differentially methylated region (DMR) sequences as set forth in Tables 2, 6, 7, 9, FIGS. 1B-1C, FIGS. 4C-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof.

5. The method of claim 1, wherein the methylation status is performed by one or more techniques selected from the group consisting of a nucleic acid amplification, polymerase chain reaction (PCR), methylation specific PCR, bisulfate pyrosequencing, single-strand conformation polymorphism (SSCP) analysis, restriction analysis, microarray technology, and proteomics.

6. A method of identifying an induced pluripotent stem (iPS) cell comprising:
comparing the methylation status of one or more nucleic acid sequences of a putative iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of a corresponding somatic cell from which the iPS cell is induced or embryonic stem (ES) cell, wherein an alteration in methylation status is indicative of the putative cell being an iPS cell.

7. The method of claim 6, wherein the one or more nucleic acid sequences are within a gene.

8. The method of claim 6, wherein the one or more nucleic acid sequences are upstream or downstream of a gene.

9. The method of claim 6, wherein the methylation status of the one or more nucleic acid sequences of the putative iPS cell are compared to the methylation status of the one or more nucleic acid sequences of a corresponding known parental somatic cell from which the iPS cell is induced.

10. The method of claim 9, wherein the one or more nucleic acid sequences are selected from the group consisting of differentially methylated region (DMR) sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof.

11. The method of claim 6, wherein the methylation status of the one or more nucleic acid sequences of the putative iPS cell are compared to the methylation status of the one or more nucleic acid sequences of a corresponding known ES cell.

12. The method of claim 11, wherein the one or more nucleic acid sequences are selected from the group consisting of differentially methylated region (DMR) sequences as set forth in Table 6, FIGS. 4C-4G, the PTPRT gene, the POU3F4 gene, the AZBP1 gene, the ZNF184 gene, the IGF1R gene, and any combination thereof.

13. The method of claim 6, wherein the alteration in methylation status is hypomethylation.

14. The method of claim 6, wherein the alteration in methylation status is hypermethylation.

15. The method according to claim 6, wherein the methylation status is performed by one or more techniques selected from the group consisting of a nucleic acid amplification, polymerase chain reaction (PCR), methylation specific PCR, bisulfite pyrosequenceing, single-strand conformation polymorphism (SSCP) analysis, restriction analysis, microarray technology, and proteomics.

16. A plurality of nucleic acid sequences, wherein the nucleic acid sequences are outside of a promoter region of a gene and outside of a CpG island, and wherein the nucleic acid sequences are up to about 2 kb in distance from a CpG island, and wherein the nucleic acid sequences are differentially methylated in the reprogramming of a somatic cell to generate an induced pluripotent stem (iPS) cell.

17. The plurality of nucleic acid sequences of claim 16, wherein the nucleic acid sequences are selected from the group consisting of the differentially methylated region (DMR) sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, and the IGF1R gene.

18. The plurality of nucleic acid sequences of claim 16, wherein the nucleic acid sequences are hypermethylated in the iPS cell as compared to the somatic cell.

19. The plurality of nucleic acid sequences of claim 16, wherein the nucleic acid sequences are hypomethylated in the iPS cell as compared to the somatic cell.

20. The plurality of nucleic acid sequences of claim 16, wherein the plurality is a microarray.

21. A plurality of nucleic acid sequences, wherein the nucleic acid sequences are outside of a promoter region of a gene and outside of a CpG island, and wherein the nucleic acid sequences are up to about 2 kb in distance from a CpG island, and wherein the methylation status of the nucleic acid sequences is altered in an induced pluripotent stem (iPS) cell as compared to an embryonic stem (ES) cell.

22. The plurality of nucleic acid sequences of claim 21, wherein the nucleic acid sequences are selected from the group consisting of the differentially methylated region (DMR) sequences as set forth in Table 7, FIGS. 4C-4G, the PTPRT gene, the POU3F4 gene, the AZBP1 gene, the ZNF184 gene, and the IGF1R gene.

23. The plurality of nucleic acid sequences of claim 21, wherein the nucleic acid sequences are hypermethylated in the iPS cell as compared to the ES cell.

24. The plurality of nucleic acid sequences of claim 21, wherein the nucleic acid sequences are hypomethylated in the iPS cell as compared to the ES cell.

25. The plurality of nucleic acid sequences of claim 21, wherein the plurality is a microarray.

26. A method for providing a methylation map of a region of genomic DNA isolated from an induced pluripotent stem (iPS) cell, comprising:
performing comprehensive high-through array-based relative methylation (CHARM) analysis on a sample of labeled, digested genomic DNA isolated from the iPS cell, thereby providing a methylation map for the iPS cell.

27. The method of claim 26, further comprises performing one or more techniques selected from the group consisting of a nucleic acid amplification, polymerase chain reaction (PCR), methylation specific PCR, bisulfite pyrosequencing, single-strand conformation polymorphism (SSCP) analysis, and restriction analysis.

28. A method of characterizing the methylation status of the nucleic acid of an induced pluripotent stem (iPS) cell, comprising:
a) hybridizing labeled and digested nucleic acid of an iPS cell to a DNA microarray comprising at least 2000 nucleic acid sequences, with the proviso that the nucleic acid sequences are outside of a promoter region of a gene and outside of a CpG island, and wherein the nucleic acid sequences are up to about 2 kb in distance from a CpG island;
b) determining a pattern of methylation from the hybridizing of (a), thereby characterizing the methylation status for the iPS cell.

29. The method of claim 28, further comprising comparing the methylation status profile to a methylation profile from hybridization of the microarray with labeled and digested nucleic acid from a parental somatic cell from which the iPS is induced.

30. The method of claim 29, wherein the one or more nucleic acid sequences are selected from the group consisting of differentially methylated region (DMR) sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, and the IGF1R gene.

31. The method of claim 28, further comprising comparing the methylation profile to a methylation profile from hybridization of the microarray with labeled and digested nucleic acid from an embryonic stem (ES) cell.

32. The method of claim 31, wherein the one or more nucleic acid sequences are selected from the group consisting of differentially methylated region (DMR) sequences as set forth in Table 7, FIGS. 4C-4G, the PTPRT gene, the POU3F4 gene, the AZBP1 gene, the ZNF184 gene, and the IGF1R gene.

33. A method of generating an induced pluripotent stem (iPS) cell comprising:
contacting a somatic cell with an agent that alters the methylation status of one or more nucleic acid sequences of the somatic cell, the one or more nucleic acid sequences being outside of a promoter region of a gene and outside of a CpG island, and wherein the nucleic acid sequences are up to about 2 kb in distance from a CpG island, and wherein the nucleic acid sequences are differentially methylated in reprogrammed somatic cells as compared with parent somatic cells, thereby generating an induced pluripotent stem (iPS) cell.

34. The method of claim 33, wherein the one or more nucleic acid sequences are selected from the group consisting of differentially methylated region (DMR) sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof.

35. The method of claim 33, further comprising detecting the methylation status profile of the one or more nucleic acid sequences of the induced iPS.

36. The method or claim 33, further comprising comparing the methylation status profile to a methylation status profile of the one or more nucleic acid sequences of a parental somatic cell from which the iPS is induced.

37. The method of claim 36, wherein the one or more nucleic acid sequences are selected from the group consisting of differentially methylated region (DMR) sequences as set forth in Tables 2, 6, 9, FIGS. 1B-1C, FIGS. 4A-4G, the BMP7 gene, the GSC gene, the TBX3 gene, the HOXD3 gene, the PTPRT gene, the POU3F4 gene, the AZBP1 gene, the ZNF184 gene, the IGF1R gene, and any combination thereof.

38. The method of claim 33, wherein the agent is a nuclear reprogramming factor.

39. The method of claim 38, wherein the nuclear reprogramming factor is a nucleic acid encoding a SOX family gene, a KLF family gene, a MYC family gene, SALL4, OCT4, NANOG, LIN28, or the expression product thereof.

40. The method of claim 38, wherein the nuclear reprogramming factor is one or more of POU5F1, OCT4, SOX2, KLF4, or C-MYC.

41. An induced pluripotent stem (iPS) cell produced using the method of claim 33.

42. A population of induced pluripotent stem (iPS) cells produced using the method of claim 33.

43. A method of treating a subject comprising:
a) obtaining a somatic cell from a subject;
b) reprogramming the somatic cell into an induced pluripotent stem (iPS) cell using the method of claim 33;
c) culturing the pluripotent stem (iPS) cell to differentiate the cell into a desired cell type suitable for treating a condition; and
d) introducing into the subject the differentiated cell, thereby treating the condition.

44. The method of claim 1, wherein methylation is determined as methylation density.

45. The method of claim 44, wherein methylation density is about 0.3 to 0.6.

46. A method of identifying an induced pluripotent stem (iPS) cell comprising:
comparing the methylation status of one or more nucleic acid sequences of a putative iPS cell, with the proviso that the one or more nucleic acid sequences are outside of a promoter region of a gene and outside of a CpG island, and wherein the methylation status is determined as methylation density of about 0.3 to 0.6.

47. The method of claim 33, wherein methylation is determined as methylation density.

48. The method of claim 47, wherein methylation density is about 0.3 to 0.6.

49. A method of generating an induced pluripotent stem (iPS) cell comprising:
contacting a somatic cell with an agent that alters the methylation status of one or more nucleic acid sequences of the somatic cell, the one or more nucleic acid sequences being outside of a promoter region of a gene and outside of a CpG island, and wherein the nucleic acid sequences are up to about 0.3 to 0.6 in methylation density, thereby generating an induced pluripotent stem (iPS) cell.

50. A method of enhancing the differentiation potential of an induced pluripotent stem (iPS) cell, comprising contacting an iPS cell with a demethylating agent, thereby reducing the epigenetic memory of the iPS cell as compared to the epigenetic memory of the iPS cell prior to contact with the demethylating agent, thereby enhancing the differentiation potential of an iPS cell as compared with a cell not contacted with a demethylating agent.

51. The method of claim 50, wherein the iPS cell is generated by contact with a nuclear reprogramming factor.

52. The method of claim 51, wherein the nuclear reprogramming factor is one or more of POU5F1, OCT4, SOX2, KLF4, or C-MYC.

53. The method of claim 50, wherein the demethylating agent is a DNA (cytosine-5)-methyltransferase 1 (DNMT1) inhibitor.

54. The method of claim 50, wherein the demethylating agent is a cytidine analog.

55. The method of claim 54, wherein the demethylating agent is agent is 5-azacytidine, 5-aza-2-deoxycytidine.

56. The method of claim 50, wherein the demethylating agent is agent is zebularine.

57. The method of claim 50, further comprising contacting the cell with a histone deacetylase (HDAC) inhibitor.

58. The method of claim 57, wherein the HDAC inhibitor is trichostatin A.

59. The method of claim 50, wherein the iPS cell is blood-derived or fibroblast derived.

60. A method of enhancing the differentiation potential of an induced pluripotent stem (iPS) cells comprising:
a) differentiating a first iPS cell generated from a first cell lineage into a cell of a second cell lineage, wherein the first and second cell lineages are different; and
b) generating a second iPS cell from the differentiated cell of a), thereby altering the epigenetic memory of the first iPS cell as compared to the epigenetic memory of the second iPS cell, thereby enhancing the differentiation potential of the second iPS cell as compared with the first iPS cell.

61. The method of claim 60, wherein the first or second iPS cell is generated by contact with a nuclear reprogramming factor.

62. The method of claim 60, further comprising contacting the first or second iPS cell with a demethylating agent.

63. The method of claim 60, further comprising contacting the first or second iPS cell with a histone deacetylase (HDAC) inhibitor.

64. The method of claim 60, wherein the first or second iPS cell is blood-derived or fibroblast derived.

65. A method of differentiating an induced pluripotent stem (iPS) cell comprising:
a) contacting an iPS cell with a demethylating agent; and
b) contacting the cell of a) with a differentiation factor,
thereby differentiating the iPS cell.

66. The method of claim 65, wherein the iPS cell is generated by contact with a nuclear reprogramming factor.

67. The method of claim 65, further comprising contacting the iPS cell with a histone deacetylase (HDAC) inhibitor.

68. A method of differentiating an induced pluripotent stem (iPS) cell comprising:
a) differentiating a first iPS cell generated from a first cell lineage into a cell of a second cell lineage, wherein the first and second cell lineages are different;
b) generating a second iPS cell from the differentiated cell of a); and
c) contacting the second iPS cell with a differentiation factor,
thereby differentiating the iPS cell.

69. The method of claim 68, wherein the first or second iPS cell is generated by contact with a nuclear reprogramming factor.

70. The method of claim 68, further comprising contacting the first or second iPS cell with a demethylating agent.

71. An induced pluripotent stem (iPS) cell produced using the method of claim 50 or 60.

72. A population of induced pluripotent stem (iPS) cells produced using the method of claim 50 or 60.

73. A method of treating a subject comprising:
a) obtaining a partially or terminally differentiated cell from a subject;
b) generating an induced pluripotent stem (iPS) cell from the cell of (a);
c) differentiating the iPS cell using the method of claim 65 or 68 to produce a desired cell type suitable for treating a condition; and
d) introducing into the subject the differentiated cell, thereby treating the condition.

74. A method of identifying the differentiation potential of an induced pluripotent stem (iPS) cell comprising:
comparing the methylation status of one or more nucleic acid sequences of an iPS cell, with the proviso that the one or more nucleic acid sequences are outside of a promoter region of a gene and outside of a CpG island, and wherein the nucleic acid sequences are up to about 2 kb in distance from a CpG island, to a known methylation status of the one or more nucleic acid sequences of a reference iPS cell or a non-induced pluripotent stem cell, wherein a similarity or a difference in methylation status between the iPS cell and the reference iPS cell or the non-induced pluripotent stem cell is indicative of the differentiation potential of the iPS cell.

75. The method of claim 74, wherein the one or more nucleic acid sequences are within a gene.

76. The method of claim 74, wherein the one or more nucleic acid sequences are upstream or downstream of a gene.

77. The method of claim 74, wherein the one or more nucleic acid sequences are selected from the group consisting of differentially methylated region (DMR) sequences as set forth in Tables 14, 15, 12, FIGS. 10, 14, 17, 18, the POU5F1 gene, the NANOG gene, the OCT4 gene, the SOX2 gene, the KLF4 gene, the C-MYC gene and any combination thereof.

78. The method of claim 74, wherein the methylation status is performed by one or more techniques selected from the group consisting of a nucleic acid amplification, polymerase chain reaction (PCR), methylation specific PCR, bisulfate pyrosequencing, single-strand conformation polymorphism (SSCP) analysis, restriction analysis, microarray technology, and proteomics.

79. The method of claim 74, wherein the reference iPS cell is blood-derived or fibroblast-derived.

80. The method of claim 74, wherein the non-induced pluripotent stem cell is a fertilized embryonic stem cell (fESC) or nuclear transfer embryonic stem cell (ntESC).

81. A method of modifying the lineage restriction of a pluripotent stem (PS) cell comprising contacting a PS cell with an agent which alters regulation of the expression or expression product of a gene known to be associated with the differentiation potential of the PS cell, thereby modifying the lineage restriction of the PS cell.

82. The method of claim 81, wherein the agent alters regulation of the expression or expression product of a gene set forth in Tables 14, 15, 12, FIGS. 10, 14, 17, 18, the POU5F1 gene, the NANOG gene, the OCT4 gene, the SOX2 gene, the KLF4 gene, the C-MYC gene and any combination thereof.

83. The method of claim 81, wherein the agent is a demethylating agent.

84. The method of claim 83, wherein the demethylating agent is a DNA (cytosine-5)-methyltransferase 1 (DNMT1) inhibitor, a cytidine analog, zebularine, a vector comprising a nucleic acid sequence encoding a gene or portion thereof, a polynucleotide, polypeptide, or small molecule.

85. The method of claim 84, wherein the gene is set forth in Tables 14, 15, 12, FIGS. 10, 14, 17, 18, the POU5F1 gene, the NANOG gene, the OCT4 gene, the SOX2 gene, the KLF4 gene, the C-MYC gene and any combination thereof.

86. The method of claim 84, wherein the polynucleotide is an antisense oligonucleotide.

87. The method of claim 86, wherein the polynucleotide is RNA.

88. The method of claim 87, wherein the RNA is selected from the group consisting of microRNA, dsRNA, siRNA, stRNA, or shRNA.

89. A method of generating a cell bank comprising:
a) identifying the differentiation potential of a plurality of pluripotent stem (PS) cells; and
b) sorting the cells of (a) by differentiation potential.

90. The method of claim 89, wherein differentiation potential is cell lineage specific.

91. The method of claim 90, wherein the PS cell is an induced pluripotent stem (iPS) cell, a fertilized embryonic stem cell (fESC), or nuclear transfer embryonic stem cell (ntESC).

92. The method of claim 91, wherein the PS cell is an iPS cell.

93. The method of claim 92, wherein (a) is performed by the method of claims 47-53.

94. A cell bank produced by the method of claim 89.

95. A method of treating a subject comprising:
a) diagnosing a subject to determine a disease or a disorder;
b) generating a plurality of pluripotent stem (PS) cells;
c) analyzing the plurality of PS cells to determine a differentiation potential for an individual stem cell of the plurality;
d) isolating an individual stem cell of (c) based on the disease or disorder of (a); and
e) introducing into the subject the stem cell of (d), thereby treating the disease or the disorder.

96. The method of claim 95, further comprising differentiating the individual stem cell of (d) using the method of claim 65 or 68 before introducing the cell into the subject to produce a desired cell type suitable for treating the disease or the disorder.

97. The method of claim 95, wherein the plurality of PS cells is generated from a partially or terminally differentiated cell isolated from the subject.

98. The method of claim 97, wherein the plurality of PS cells are induced PS cells.


SEQUENCE LISTING (88 sequences)

SEQ ID NO: 1 - DNA, 25 bp, Artificial Sequence
  Primer
tttggtttggaaatgtattaatata

SEQ ID NO: 2 - DNA, 30 bp, Artificial Sequence
  Primer
taacaataccaaaaaatactaaaactacta

SEQ ID NO: 3 - DNA, 30 bp, Artificial Sequence
  Primer
ttttggttttaaaataataaagtaattatt

SEQ ID NO: 4 - DNA, 25 bp, Artificial Sequence
  Primer
aactcaaacaaacatatacaatacc

SEQ ID NO: 5 - DNA, 22 bp, Artificial Sequence
  Primer
gttgttattaatttaatttatt

SEQ ID NO: 6 - DNA, 30 bp, Artificial Sequence
  Primer
gatttaagttattatgttttagggtagata

SEQ ID NO: 7 - DNA, 25 bp, Artificial Sequence
  Primer
aaaacaatattccaaataaaaaaaa

SEQ ID NO: 8 - DNA, 29 bp, Artificial Sequence
  Primer
ttaggtttaaagttatagggtagttgatg

SEQ ID NO: 9 - DNA, 25 bp, Artificial Sequence
  Primer
tttaacatctttacaaaaacaaaac

SEQ ID NO: 10 - DNA, 21 bp, Artificial Sequence
  Primer
gtaatttattagtgattgttt

SEQ ID NO: 11 - DNA, 27 bp, Artificial Sequence
  Primer
ttaaagagtaaataaagaaaaggtgtt

SEQ ID NO: 12 - DNA, 25 bp, Artificial Sequence
  Primer
aatcctaaaaatccaaacataattc

SEQ ID NO: 13 - DNA, 30 bp, Artificial Sequence
  Primer
tgaaagtaattagatttgtattttaatagt

SEQ ID NO: 14 - DNA, 26 bp, Artificial Sequence
  Primer
aattttatatcctctaaaacataacc

SEQ ID NO: 15 - DNA, 22 bp, Artificial Sequence
  Primer
gatggaatatttttgattttgt

SEQ ID NO: 16 - DNA, 25 bp, Artificial Sequence
  Primer
ttaggatttagggtttttgtttttt

SEQ ID NO: 17 - DNA, 30 bp, Artificial Sequence
  Primer
tatcatcttcctaaatatttcacaaatatt

SEQ ID NO: 18 - DNA, 25 bp, Artificial Sequence
  Primer
gtgggtaggaagaagttttaaggtt

SEQ ID NO: 19 - DNA, 25 bp, Artificial Sequence
  Primer
aactcatttctcaaataaaaaaccc

SEQ ID NO: 20 - DNA, 23 bp, Artificial Sequence
  Primer
ttattagagttttttagtagatt

SEQ ID NO: 21 - DNA, 25 bp, Artificial Sequence
  Primer
gtagattggtttttttgtatttttg

SEQ ID NO: 22 - DNA, 30 bp, Artificial Sequence
  Primer
tataaactcttcaaatttcttttaatatct

SEQ ID NO: 23 - DNA, 24 bp, Artificial Sequence
  Primer
gatttatttggttagagggtttgg

SEQ ID NO: 24 - DNA, 25 bp, Artificial Sequence
  Primer
aaaaaacttttcccacttaaaaaac

SEQ ID NO: 25 - DNA, 24 bp, Artificial Sequence
  Primer
gatttatttggttagagggtttgg

SEQ ID NO: 26 - DNA, 25 bp, Artificial Sequence
  Primer
aaggttatagggattttggtttatt

SEQ ID NO: 27 - DNA, 25 bp, Artificial Sequence
  Primer
ccacaacaactacatatttttaaaa

SEQ ID NO: 28 - DNA, 25 bp, Artificial Sequence
  Primer
atttttgtgtgtatgtgtttttgtg

SEQ ID NO: 29 - DNA, 25 bp, Artificial Sequence
  Primer
ctctacacaacctaaccaaattttt

SEQ ID NO: 30 - DNA, 25 bp, Artificial Sequence
  Primer
atttttgtgtgtatgtgtttttgtg

SEQ ID NO: 31 - DNA, 24 bp, Artificial Sequence
  Primer
tttttgataaattgatgggatgtg

SEQ ID NO: 32 - DNA, 25 bp, Artificial Sequence
  Primer
aaccctaaaactaaccaccaaaaac

SEQ ID NO: 33 - DNA, 25 bp, Artificial Sequence
  Primer
taagatgaaaagtggaaagaaatag

SEQ ID NO: 34 - DNA, 25 bp, Artificial Sequence
  Primer
ataaaaactctaaacccaaccatca

SEQ ID NO: 35 - DNA, 26 bp, Artificial Sequence
  Primer
gaagattttatagttattttaaatag

SEQ ID NO: 36 - DNA, 27 bp, Artificial Sequence
  Primer
aaaagaaaatttttaagttataaaatt

SEQ ID NO: 37 - DNA, 30 bp, Artificial Sequence
  Primer
aaatcaaaatccatatctcatttaatctaa

SEQ ID NO: 38 - DNA, 25 bp, Artificial Sequence
  Primer
ttgggagagttttaaagttatttgg

SEQ ID NO: 39 - DNA, 25 bp, Artificial Sequence
  Primer
taactccaatccaaaattttctctc

SEQ ID NO: 40 - DNA, 25 bp, Artificial Sequence
  Primer
tgggagagttttaaagttatttgga

SEQ ID NO: 41 - DNA, 24 bp, Artificial Sequence
  Primer
gtggtttgggaagatatgaatttt

SEQ ID NO: 42 - DNA, 25 bp, Artificial Sequence
  Primer
aaaaataaaaacccccttttcttac

SEQ ID NO: 43 - DNA, 25 bp, Artificial Sequence
  Primer
aaggttttttatttgtttttgatta

SEQ ID NO: 44 - DNA, 22 bp, Artificial Sequence
  Primer
aaaatcctaaaccctccacttc

SEQ ID NO: 45 - DNA, 24 bp, Artificial Sequence
  Primer
aggttttttatttgtttttgatta

SEQ ID NO: 46 - DNA, 25 bp, Artificial Sequence
  Primer
gttgttttgttttggttttggatat

SEQ ID NO: 47 - DNA, 24 bp, Artificial Sequence
  Primer
caaaaaaccttcattttcaacctt

SEQ ID NO: 48 - DNA, 25 bp, Artificial Sequence
  Primer
tgaggagtggttttagaaataattg

SEQ ID NO: 49 - DNA, 24 bp, Artificial Sequence
  Primer
aatcctctcacccctaccttaaat

SEQ ID NO: 50 - DNA, 25 bp, Artificial Sequence
  Primer
tgaggagtggttttagaaataattg

SEQ ID NO: 51 - DNA, 25 bp, Artificial Sequence
  Primer
gagggtgtagtgttaataggttttg

SEQ ID NO: 52 - DNA, 30 bp, Artificial Sequence
  Primer
gtaatagagaaaaatttgttttaaaattaa

SEQ ID NO: 53 - DNA, 25 bp, Artificial Sequence
  Primer
ctacaaacataaaaaaatcaaacct

SEQ ID NO: 54 - DNA, 25 bp, Artificial Sequence
  Primer
tttaagtaggatataggtttttttt

SEQ ID NO: 55 - DNA, 25 bp, Artificial Sequence
  Primer
actaccaaaatctctatttatacac

SEQ ID NO: 56 - DNA, 25 bp, Artificial Sequence
  Primer
tttaagtaggatataggtttttttt

SEQ ID NO: 57 - DNA, 25 bp, Artificial Sequence
  Primer
tttaatgtgaagagtaagtaagaaa

SEQ ID NO: 58 - DNA, 28 bp, Artificial Sequence
  Primer
agatgtgagtttttgtagggagtgtata

SEQ ID NO: 59 - DNA, 25 bp, Artificial Sequence
  Primer
catattcttaatccctaaaccccat

SEQ ID NO: 60 - DNA, 29 bp, Artificial Sequence
  Primer
tttagttgggagaaaaagagtttattaaa

SEQ ID NO: 61 - DNA, 24 bp, Artificial Sequence
  Primer
caaacctaactacacacctacacc

SEQ ID NO: 62 - DNA, 25 bp, Artificial Sequence
  Primer
tttttagtatttgggttttgtttta

SEQ ID NO: 63 - DNA, 26 bp, Artificial Sequence
  Primer
taattttgtatggagagtttggtttg

SEQ ID NO: 64 - DNA, 26 bp, Artificial Sequence
  Primer
ccccaattatatttaattaccttcac

SEQ ID NO: 65 - DNA, 25 bp, Artificial Sequence
  Primer
tttgtagaagtaaaggagtgtgata

SEQ ID NO: 66 - DNA, 27 bp, Artificial Sequence
  Primer
tcactacaataactcctataaaaaaaa

SEQ ID NO: 67 - DNA, 26 bp, Artificial Sequence
  Primer
tggtagatgttttagtagggttttag

SEQ ID NO: 68 - DNA, 28 bp, Artificial Sequence
  Primer
tgggagataattattttttagaaagtga

SEQ ID NO: 69 - DNA, 27 bp, Artificial Sequence
  Primer
tcccaaactttaacctatttctctaca

SEQ ID NO: 70 - DNA, 25 bp, Artificial Sequence
  Primer
ttgattttaaagggttggaaaatat

SEQ ID NO: 71 - DNA, 25 bp, Artificial Sequence
  Primer
aaaacttaaccttaaaactcctaca

SEQ ID NO: 72 - DNA, 23 bp, Artificial Sequence
  Primer
aagttttagttgttttagaaata

SEQ ID NO: 73 - DNA, 25 bp, Artificial Sequence
  Primer
tttggttgtatttttaggaattatt

SEQ ID NO: 74 - DNA, 20 bp, Artificial Sequence
  Primer
aaaaacaaccccaaataacc

SEQ ID NO: 75 - DNA, 25 bp, Artificial Sequence
  Primer
ttgtgaggatttttatatttttttt

SEQ ID NO: 76 - DNA, 24 bp, Artificial Sequence
  Primer
accaaacccaaaaactcaactaat

SEQ ID NO: 77 - DNA, 21 bp, Artificial Sequence
  Primer
ttttttgatttaatatttaga

SEQ ID NO: 78 - DNA, 31 bp, Artificial Sequence
  Primer
agctgctgaagcagaagaggatcatctcatt

SEQ ID NO: 79 - DNA, 17 bp, Artificial Sequence
  Primer
gttgtcggcttcctcca

SEQ ID NO: 80 - DNA, 30 bp, Artificial Sequence
  Primer
aaccaaaggatgaagtgcaagcggtccaag

SEQ ID NO: 81 - DNA, 18 bp, Artificial Sequence
  Primer
ttgggttggtccaagtct

SEQ ID NO: 82 - DNA, 20 bp, Artificial Sequence
  Primer
tgaagtgtgacgtggacatc

SEQ ID NO: 83 - DNA, 19 bp, Artificial Sequence
  Primer
ggaggagcaatgatcttga

SEQ ID NO: 84 - DNA, 12 bp, Mus musculus
gcacaygaacat

SEQ ID NO: 85 - DNA, 13 bp, Mus musculus
acaggcygagagg

SEQ ID NO: 86 - DNA, 46 bp, Mus musculus
ggtgygatggggcatcygagcaactggtttgtgaggtgtcyggtga

SEQ ID NO: 87 - DNA, 24 bp, Mus musculus
tcacttgygttaaaaagcygcact

SEQ ID NO: 88 - DNA, 43 bp, Mus musculus
aagcaagaaaygctgagtgctgaaaggaaagcygtgtataaac